(19) United States
(12) Patent Application Publication
(10) Pub. No.: US 2026/0278785 A1
(43) Pub. Date: Sep. 17, 2026

(54) TRAINING OF A MACHINE LEARNING MODEL FOR USE IN MEDICAL IMAGING APPLICATIONS BASED ON COMBINATIONS OF INCOMPLETE SAMPLE SETS AND SAMPLE IMAGES SIMULATED THEREFROM

(71) Applicant: BRACCO IMAGING SPA, Milano (IT)

(72) Inventors: Giovanni Valbusa, Colleretto Giacosa (IT); Sonia Colombo Serra, Colleretto Giacosa (IT); Alberto Fringuello Mingo, Colleretto Giacosa (IT); Fabio Tedoldi, Colleretto Giacosa (IT); Davide Bella, Colleretto Giacosa (IT)

(73) Assignee: BRACCO IMAGING SPA, Milano (IT)

(21) Appl. No.: 19/472,477

(22) PCT Filed: Apr. 11, 2024
(86) PCT No.: PCT/EP2024/059876

§ 371 Date: Oct. 3, 2025

(30) Foreign Application Priority Data
Apr. 14, 2023 (EP) 23168015.8

(51) Int. Cl. G06T 7/00; G06V 10/774; G16H 30/40

(52) U.S. Cl. CPC G06T 7/0012; G06V 10/774; G16H 30/40; G06T 2207/20081; G06T 2207/20084

(57) ABSTRACT

A solution is proposed for training a machine learning model (305) for use in medical imaging applications. A corresponding method (400) comprises providing (403-410) incomplete sample sets for each imaging procedure, each comprising a sample target image (or more) for a sample target-dose of a contrast agent and a sample baseline image (or more). A sample source image (or more) is simulated (411-445; 450-456) from each of the incomplete sample sets (or a part thereof) to mimic the contrast agent at a sample source-dose lower than the sample target-dose. One or more complete sample sets are generated (446; 457) for each imaging procedure by combining the incomplete sample sets with the sample source images that have been simulated from other incomplete sample sets. The machine learning model (305) is then trained (458-473) using the complete sample sets. In addition, a method of using the machine learning model in a medical imaging application is proposed. A computer programs (300) and a computer program products for implementing the method (400) are proposed. Moreover, a computing system (130) for performing the method (400) is proposed. A corresponding medical method is further proposed.

### TECHNICAL FIELD

The present disclosure relates to the field of machine learning. More specifically, this disclosure relates to machine learning for use in medical imaging applications.

### BACKGROUND ART

The background of the present disclosure is hereinafter introduced with the discussion of techniques relating to its context. However, even when this discussion refers to documents, acts, artifacts and the like, it does not suggest or represent that the discussed techniques are part of the prior art or are common general knowledge in the field relevant to the present disclosure.

Imaging techniques are commonplace in medical applications to inspect body-parts of patients by physicians through images providing visual representations thereof (typically, in a substantially non-invasive manner even if the body-parts are not visible directly). For this purpose, a contrast agent is typically administered to a patient undergoing an imaging procedure for enhancing contrast of a (biological) target of interest, for example, a lesion, so as to make it more conspicuous in the images.

In this context, it has also been proposed to use a reduced-dose of the contrast agent; the reduced-dose is lower than a full-dose of the contrast agent that is standard in clinical practice. For this purpose, during the imaging procedure a zero-dose image of the body-part is acquired before administration of the contrast agent and one or more reduced-dose images of the body-part are acquired after administration of the reduced-dose of the contrast agent to the patient. Corresponding full-dose images of the body-part, mimicking administration of the full-dose of the contrast agent to the patient, are then simulated from the zero-dose image and the corresponding reduced-dose images by means of a Deep Learning Network (DLN); the deep learning network restores the contrast enhancement from its level in the reduced-dose images (being inadequate because of the reduced-dose of the contrast agent) to the desired level that would have been provided by the contrast agent at the full-dose. The deep learning network is trained by using sample sets each comprising a zero-dose image, a reduced-dose image and a full-dose image of a body-part of the same type being acquired before administration of the contrast agent, after administration of the reduced-dose of the contrast agent and after administration of the full-dose of the contrast agent to a corresponding patient (or two or more zero-dose images acquired under different acquisition conditions or two or more reduced-dose images acquired with different reduced-doses of the contrast agent).

However, a number of the sample sets of (zero-dose/reduced-dose/full-dose) images that are available for training the deep learning network is relatively low. This is mainly due to the fact that the reduced-dose images are not normally acquired in standard clinical practice (since they would require corresponding modifications of the imaging procedures for the multiple administrations of the contrast agent at the reduced-dose and at the full-dose). The low number of the sample sets reduces a quality of the training of the deep learning network. This has a negative impact on the robustness of the deep learning network, and particularly on its capability of predicting the full-dose images.

Simulation of the reduced-dose images has also been proposed.

For example, WO-A-2021/061710 discloses including enhanced data sets obtained from simulations to input data used to train a deep learning network for use to improve quality of images acquired with reduced-dose of contrast agent. For this purpose, image data from a clinical database can be used to generate low-quality image data that mimic image data acquired with a reduced-dose of the contrast agent; this result is achieved by adding artifacts to the original image data.

Document “Johannes Haubold et al., Contrast agent dose reduction in computed tomography with deep learning using a conditional generative adversarial network, European Radiology (2021) 31:6087-6095” instead discloses the simulation of images with reduced Ioding-based contrast media (ICM) to validate the possibility of virtually enhancing the ICM. For this purpose, dual-energy computed tomography (CT) images based on the ICM are acquired. Isolated ICM images are generated encoding the distribution of the ICM, and they are used to create virtual non-contrast (VCN) images. Dual-energy CT images corresponding to the reduced ICM (by 50% or 80%) are simulated via proportional subtraction. Pairs of input images and target images are obtained by combining the reduced ICM images and the isolated ICM images, respectively, with the VCN images. A generative adversarial network (to be used to simulate ICM enhancement for validating it) is trained with the pairs of input/target images.

However, the simulation of the full-dose images from the zero-dose image and the corresponding reduced-dose images by means of a deep learning network remains challenging. Particularly, the obtained result may suffer from artefacts, inhomogeneous texture and/or signal intensity changes.

### SUMMARY

A simplified summary of the present disclosure is herein presented in order to provide a basic understanding thereof; however, the sole purpose of this summary is to introduce some concepts of the disclosure in a simplified form as a prelude to its following more detailed description, and it is not to be interpreted as an identification of its key elements nor as a delineation of its scope.

In general terms, the present disclosure is based on the idea of decorrelating the images used to train the machine learning model.

Particularly, an aspect provides a method for training a machine learning model for use in medical imaging applications. The method comprises providing incomplete sample sets for each imaging procedure, each comprising a sample target image (or more) for a sample target-dose of a contrast agent and a sample baseline image (or more). A sample source image (or more) is simulated from each of the incomplete sample sets (or a part thereof) to mimic the contrast agent at a sample source-dose lower than the sample target-dose. One or more complete sample sets are generated for each imaging procedure by combining the incomplete sample sets with the sample source images that have been simulated from other incomplete sample sets. The machine learning model is then trained using the complete sample sets.

A further aspect provides a method of using the machine learning model in a medical imaging application.

A further aspect provides a computer program for implementing each method.

A further aspect provides a corresponding computer program product.

A further aspect provides a computing system for implementing each method.

A further aspect provides a corresponding medical method.

More specifically, one or more aspects of the present disclosure are set out in the independent claims and advantageous features thereof are set out in the dependent claims, with the wording of all the claims that is herein incorporated verbatim by reference (with any advantageous feature provided with reference to any specific aspect that applies mutatis mutandis to every other aspect).

### BRIEF DESCRIPTION OF THE DRAWINGS

The solution of the present disclosure, as well as further features and the advantages thereof, will be best understood with reference to the following detailed description thereof, given purely by way of a non-restrictive indication, to be read in conjunction with the accompanying drawings (wherein, for the sake of simplicity, corresponding elements are denoted with equal or similar references and their explanation is not repeated, and the name of each entity is generally used to denote both its type and its attributes, such as value, content and representation). Particularly:

FIG. 1 shows a schematic block diagram of an infrastructure that may be used to practice the solution according to an embodiment of the present disclosure,

FIG. 2 shows the general principles of the solution according to an embodiment of the present disclosure,

FIG. 3 shows the main software components that may be used to implement the solution according to an embodiment of the present disclosure,

FIG. 4A-FIG. 4D show an activity diagram describing the flow of activities relating to an implementation of the solution according to an embodiment of the present disclosure, and

FIG. 5A-FIG. 5C show representative examples of experimental results relating to the solution according to an embodiment of the present disclosure.

### DETAILED DESCRIPTION

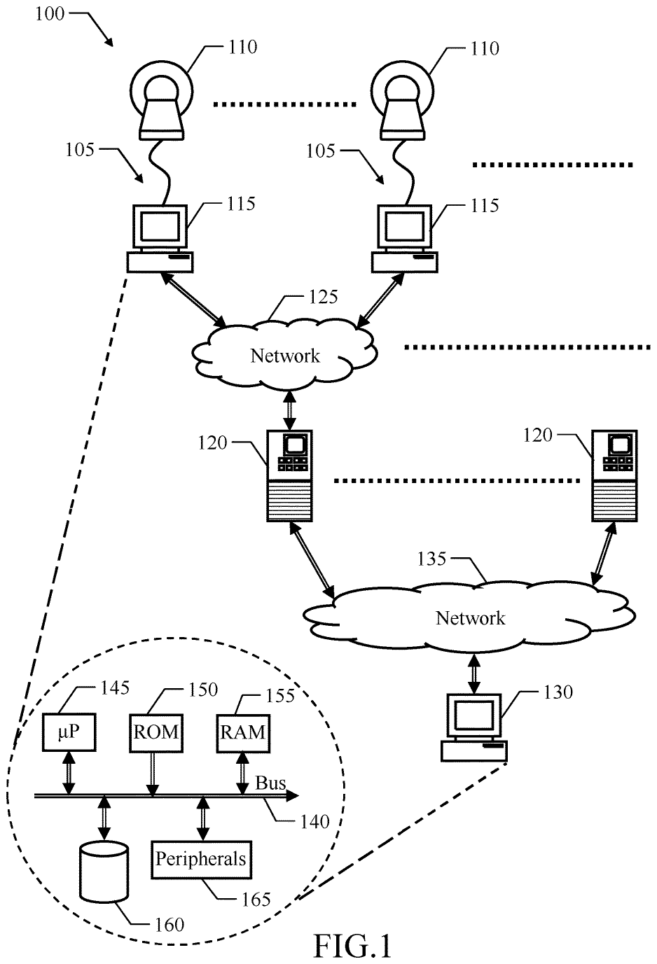

With reference in particular to FIG. 1, a schematic block diagram is shown of an infrastructure 100 that may be used to practice the solution according to an embodiment of the present disclosure.

The infrastructure 100 comprises the following components.

One or more (medical) imaging systems 105 comprise corresponding scanners 110 and control computing systems, or simply control computers 115. Each scanner 110 is used to acquire images representing body-parts of subjects during corresponding (medical) imaging procedures based on administration thereto of a contrast agent for enhancing contrast of a corresponding (biological) target, such as a lesion. The subjects may be either animals in pre-clinical studies or persons (i.e., human beings) in clinical applications. For example, the scanner 110 is of Magnetic Resonance Imaging (MRI) type. In this case, not represented in the figure, the (MRI) scanner 110 has a gantry for receiving a subject; the gantry houses a superconducting magnet (for generating a very high stationary magnetic field), multiple sets of gradient coils for different axes (for adjusting the stationary magnetic field) and an RF coil (with a specific structure for applying magnetic pulses to a type of body-part and for receiving corresponding response signals). As an alternative, the scanner 110 is of Computed Tomography (CT) type. In this case, again not represented in the figure, the (CT) scanner 110 has a gantry for receiving a subject; the gantry houses an X-ray generator, an X-ray detector and a motor for rotating them around a body-part of the subject. The corresponding control computer 115, for example, a Personal Computer (PC), is used to control operation of the scanner 110. For this purpose, the control computer 115 is coupled with the scanner 110. For example, in case the scanner 110 is of MRI type the control computer 115 is arranged outside a scanner room used to shield the scanner 110 and it is coupled with it via a cable passing through a penetration panel, whereas in case the scanner 110 is of CT type the control computer 110 is arranged close to it.

The imaging systems 105 are installed at one or more facilities, which may be either lab facilities (for example, universities) in case of imaging procedures of pre-clinical type or health facilities (for example, hospitals) in case of imaging procedures of clinical type. Each (lab/health) facility comprises one or more of the imaging systems 105. Moreover, the facility may comprise a central computing system, or simply central server 120, which communicates with the control computers 115 of its imaging systems 105 over a network 125, for example, a Local Area network (LAN) of the facility. The central server 120 gathers information about the imaging procedures that have been performed by at least part of the imaging systems 105 from their control computers 115 over the network 125; particularly, for each imaging procedure this comprises a sequence of images representing the corresponding body-part and additional information relating to the imaging procedure, for example, identification of the subject, result of the imaging procedure, acquisition parameters of the imaging procedure and so on.

A configuration computing device 130, or simply configuration computer 130 (or more) is used to configure the control computers 115 of the imaging systems 105. The configuration computer 130 may communicate with the central servers 120 of the facilities over a network 135, for example, based on the Internet. The configuration computer 130 (anonymously) collects the information relating to the imaging procedures that have been performed in at least part of the facilities from the corresponding central servers 120 over the network 135 (alternatively, not shown in the figure, the same information may be collected from the corresponding control computers 115 over the network 135 or from the central servers 120 and/or the control computers 115 via removable storage units, such as of USB type). The information so collected by the configuration computer 130 is used to configure at least part of the control computers 115 of the imaging systems 105, either on-site in the facilities after their shipping or in a factory before their shipping to the facilities (as described in detail in the following).

Each computing machine implementing the control computers 115, the central servers 120 and the configuration computer 130 comprises several units that are connected among them through a bus structure 140. Particularly, a microprocessor (μP) 145, or more, provides a logic capability of the computing machine 115,120,130. A non-volatile memory (ROM) 150 stores basic code for a bootstrap of the computing machine 115,120,130 and a volatile memory (RAM) 155 is used as a working memory by the microprocessor 145. The computing machine 115,120,130 is provided with a mass-memory 160 for storing programs and data, for example, a Solid-State Disk (SSD). Moreover, the computing machine 115,120,130 comprises a number of controllers 165 for peripherals, or Input/Output (I/O), units. Particularly, as far as relevant to the present disclosure, the peripherals comprise a keyboard, a mouse, a monitor, a network adapter (NIC) for connecting to the corresponding network 125,135, a drive for reading/writing removable storage units (such as USB keys) and, for each control computer 115, a trackball and corresponding drives for relevant units of its scanner 110.

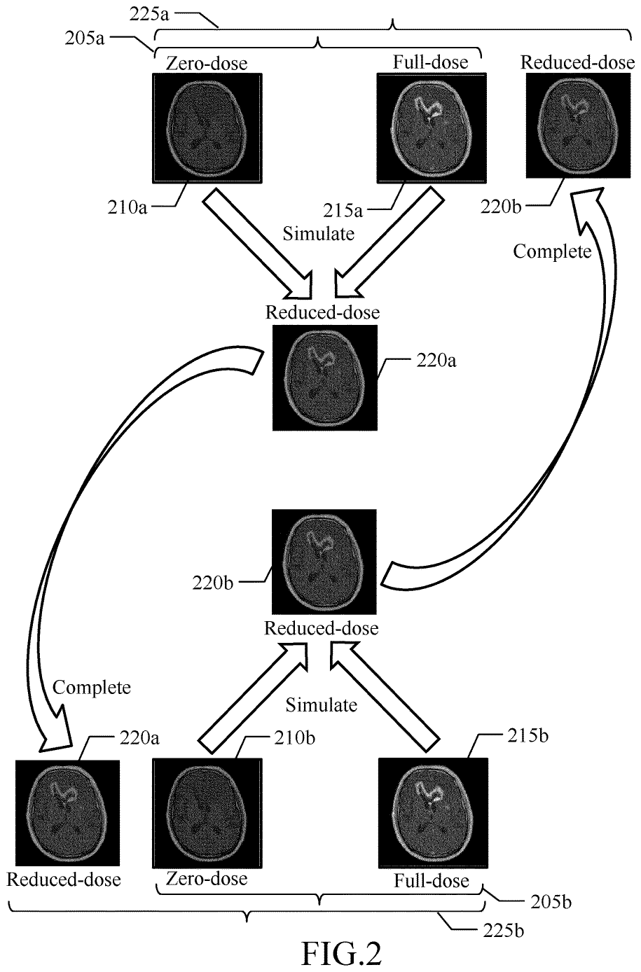

With reference now to FIG. 2, the general principles are shown of the solution according to an embodiment of the present disclosure.

This relates to a training of an (operative) machine learning model to be used in medical imaging applications to mimic an increase of a dose of a contrast agent that is administered to corresponding patients. For this purpose, (sample) images are collected from a plurality of (sample) imaging procedures relating to corresponding (sample) body-parts of (sample) subjects. Particularly, a plurality of incomplete sample sets are provided for each imaging procedure. Each incomplete sample set comprises a sample target image (or more) and a sample baseline image (or more). The sample target image represents the corresponding body-part of the subject to which a contrast agent has been administered at a sample target-dose; the sample baseline image instead represents the corresponding body-part under a different condition (for example, without the contrast agent). For each of the incomplete sample sets (or a part thereof) of each imaging procedure, a sample source image (or more) is simulated (or synthesized) from the other sample images of the incomplete sample set, i.e., the sample baseline image and the sample target image (for example, analytically). The sample source image represents the corresponding body-part mimicking administration to the corresponding subject of the contrast agent at a sample source-dose. The sample source-dose is lower than the sample target-dose, with a ratio between the sample source-dose and the sample target-dose equal to a downscaling factor lower than 1 (for example, 1/10).

In the solution according to an embodiment of the present disclosure, one or more complete sample sets are generated for each imaging procedure. Each complete sample set comprises the sample baseline image and the sample target image of an incomplete sample set of the imaging procedure and the sample source image that has been simulated from another (different) incomplete sample set of the imaging procedure.

The complete sample sets are then used to train the machine learning model, so as to optimize its capability of predicting the sample target image (ground truth) of each complete sample set from the sample baseline image and the sample source image of the complete sample set (for example, by using part of the complete sample sets to form training sets of the machine learning model and another part of the complete sample sets to form verification sets of the machine learning model).

The machine learning model so trained may be used in several ways in the medical imaging applications. Particularly, during each (operative) imaging procedure the corresponding scanner acquires (operative) images representing an (operative) body-part of an (operative) patient under examination. The operative images comprise an operative baseline image (or more) and one or more operative administration images. The operative administration images are acquired from the patient to which the contrast agent has been administered at an operative administration-dose; the operative baseline image is acquired from the body-part under a different condition (for example, again without the contrast agent). The control computer associated with the scanner uses the machine learning model to simulate (or synthesize) one or more operative simulation images each from the operative baseline image and a corresponding operative administration image. The operative simulation images represent the body-part mimicking administration to the patient of the contrast agent at an operative simulation-dose. The operative simulation-dose is higher than the operative administration-dose, with a ratio between the operative simulation-dose and the operative administration-dose equal to an upscaling factor corresponding to the inverse of the downscaling factor of the machine learning model (for example, equal thereto). Therefore, the operative administration-dose and the operative administration images are also referred to as operative low-dose and operative low-dose images, respectively, and the operative simulation-dose and the operative simulation images are also referred to as operative high-dose and operative high-dose images, respectively. A representation of the body-part based on the operative simulation images is then output (for example, by displaying them) to a physician in charge of the imaging procedure.

The above-described solution significantly improves the training of the machine learning model, with a positive impact on its robustness. This is due to the fact that the proposed combination of the sample baseline/target images with the sample source images that are simulated from different sample baseline/target images removes (or at least substantially reduces) their correlation. As a result, the machine learning model is prevented from learning corresponding unwanted features (such as stochastic fluctuations) that might be transferred to the operative simulation images that are simulated by it. This reduces artefacts, inhomogeneous texture and/or signal intensity changes in the operative simulation images.

As a result, the machine learning model is capable of predicting the operative simulation images with a relatively high accuracy. This has a beneficial effect on the quality of the medical imaging applications, for example, substantially reducing the risk of false positives/negatives and wrong follow-up in diagnostic applications, the risk of reduced effectiveness of therapies or of damages to healthy tissues in therapeutic applications and the risk of incomplete resection of lesions or excessive removal of healthy tissues is surgical applications.

The above-described solution may be applied directly to sample images that are acquired in pre-clinical imaging procedures. Moreover, it may also be extended to sample images that are acquired in (prospective) clinical imaging procedures by simply acquiring one or more further images without affecting the standard of care (i.e., dose and injection protocol of the contrast agent).

Particularly, the sample baseline images may have been acquired from the corresponding subjects to which the contrast agent has never been administered or a relatively long time has elapsed from a previous administration of the contrast agent ensuring that it has been substantially cleared, so that the corresponding body-parts are without the contrast agent (or at least with no significant amount thereof); in this case, the sample baseline images are referred to as sample zero-dose images. The sample target-dose of the contrast agent may be equal to a value that is standard in clinical practice (with the sample target images being acquired at any time after administration of the contrast agent, either the same or different with respect to a value that is standard in clinical practice); in this case, the sample target-dose and the sample target images are referred to as full-dose and sample full-dose images, respectively. Therefore, the sample source-dose of the contrast agent is reduced with respect to the value that is standard in clinical practice; in this case, the sample source-dose and the sample source images are referred to as reduced-dose and sample reduced-dose images, respectively.

For the sake of simplicity, in the following reference will be made to this implementation of the sample baseline images, the sample target-dose, the sample target-dose images, the sample source-dose and the sample source-dose images (however, with the same considerations that apply to any other implementation thereof).

For example, FIG. 2 shows a simple scenario wherein two incomplete sample sets 205a and 205b are provided for a same imaging procedure: the incomplete sample set 205a comprises a sample zero-dose image 210a and a sample full-dose image 215a, and the incomplete sample set 205b comprises a sample zero-dose image 210b and a sample full-dose image 215b. A sample reduced-dose image 220a is simulated from the incomplete sample set 205a (sample zero-dose image 210a and sample full-dose image 215a) and a sample reduced-dose image 220b is simulated from the incomplete sample set 205b (sample zero-dose image 210b and sample full-dose image 215b). A complete sample set 225a is generated to comprise the sample zero-dose image 210a and the sample full-dose image 215a (of the incomplete sample set 205a) with the addition of the sample reduced-dose image 220b (simulated from the incomplete sample set 205b), whereas a complete sample set 225b is generated to comprise the sample zero-dose image 210b and the sample full-dose image 215b (of the incomplete sample set 205b) with the addition of the sample reduced-dose image 220a (simulated from the incomplete sample set 205a).

With reference instead to the use of the machine learning model in the medical imaging applications, as above the operative baseline image may have been acquired from the patient to which the contrast agent has never been administered or a relatively long time has elapsed from a previous administration of the contrast agent ensuring that it has been substantially cleared, so that the body-part is without the contrast agent (or at least with no significant amount thereof); in this case, the operative baseline image is referred to as operative zero-dose image.

The operative administration-dose and the operative simulation-dose of the contrast agent may be equal to its reduced-dose and full-dose, respectively; in this case, the operative administration images and the operative simulation images are referred to as operative reduced-dose images and operative full-dose images, respectively. As a result, it is possible to restore the contrast enhancement that would have been obtained normally with the administration of the contrast agent at the full-dose (especially advantageous when this may be dangerous for the patient), if not even increasing it by reducing motion/aliasing artifacts that might be caused by the actual administration of the contrast agent at the full-dose.

The operative administration-dose of the contrast agent may also be equal to its full-dose, so that the operative simulation-dose is boosted (or increased/augmented) with respect thereto; in this case, the operative administration images are referred to as operative full-dose images, and the operative simulation-dose and the operative simulation images are referred to as operative boosted-dose and operative boosted-dose images, respectively. As a result, it is possible to increment the contrast enhancement as if the operative boosted-dose images were acquired with the administration of the contrast agent at a (virtual) dose higher than the one attainable in current clinical practice (especially advantageous when the contrast enhancement is too poor), at the same time without any impact on the standard of care. As a further improvement, operative combined images may be generated each from the operative baseline image, an operative full-dose image and the corresponding operative boosted-dose image by applying High Dynamic Range (HDR) techniques (for example, by giving more importance to the contribution of the boosted-dose image thereto). This makes the target more conspicuous at the same time maintaining it well contextualized on a morphology of the body-part.

It is also possible to provide multiple versions of the machine learning model that have been trained with different values of the downscaling factor. In this case, in the medical imaging applications the value of the upscaling factor may be selected among corresponding values. This adds further flexibility, allowing the physician to verify the effects of the different values of the upscaling factor in real-time, and then to select the one that provides the best contrast enhancement.

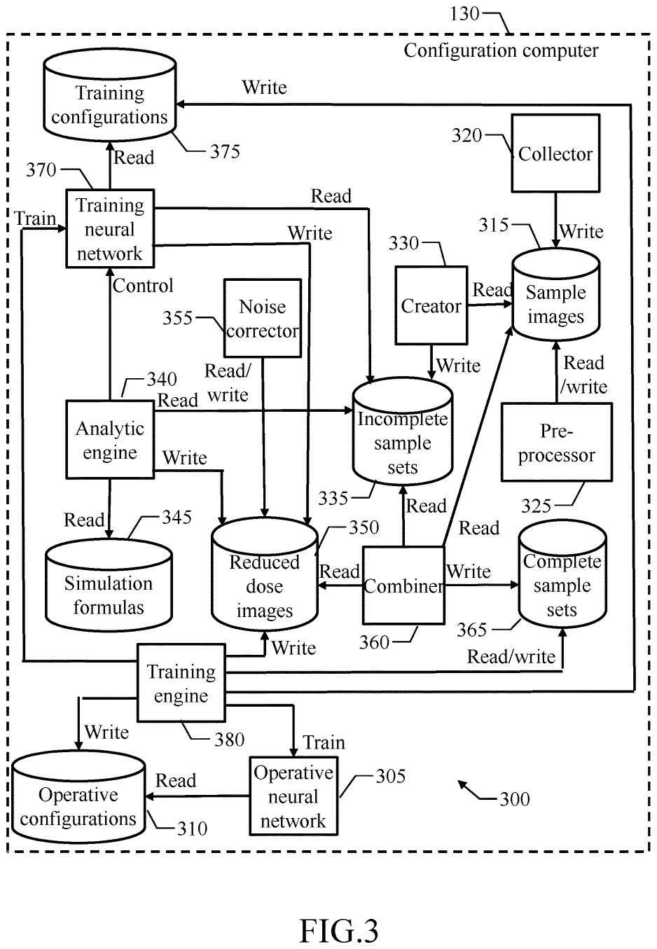

With reference now to FIG. 3, the main software components are shown that may be used to implement the solution according to an embodiment of the present disclosure.

All the software components (programs and data) are denoted as a whole with the reference 300. The software components 300 are typically stored in the mass memory and loaded (at least in part) into the working memory of the configuration computer 130 when the programs are running, together with an operating system and other application programs not directly relevant to the solution of the present disclosure (thus omitted in the figure for the sake of simplicity). The programs are initially installed into the mass memory, for example, from removable storage units or from the network. In this respect, each program may be a module, segment or portion of code, which comprises one or more executable instructions for implementing the specified logical function.

The configuration computer 130 stores a copy of the machine learning model to be trained. For example, the machine learning model is implemented by an (operative) neural network 305 (to which reference will be made in the following, with the same considerations that apply to any other implementation thereof).

Basically, machine learning is used to perform a specific task (in this case, simulating the operative simulation images) without using explicit instructions but inferring how to do so automatically from examples (by exploiting a corresponding model that has been learnt from them). In an embodiment of the present disclosure, there is applied a deep learning technique, which is a branch of machine learning based on deep neural networks. Basically, a neural network is a data processing system inspired by operation of human brain. The neural network comprises basic processing elements (neurons), which perform operations based on corresponding weights; the neurons are connected via unidirectional channels (synapses), which transfer data among them. The neurons are organized in layers performing different operations, always comprising an input layer and an output layer for receiving input data and for providing output data, respectively, of the neural network. Particularly, a deep neural network has one or more hidden layers being arranged in succession between the input layer and the output layer along a processing direction of the deep neural network. In an embodiment of the present disclosure, the neural network is a Convolutional Neural Network (CNN), i.e., a specific type of deep neural network wherein one or more of its hidden layers perform (cross) convolution operations. Particularly, the neural network is an autoencoder (encoder-decoder) convolutional neural network, which comprises an encoder that compacts the data in a denser form (in a so-called latent space), which data so compacted are used to perform the desired operations, and a decoder that expands the result so obtained into a required more expanded form. More in detail, the input layer is configured to receive two input images (i.e., a sample baseline image and a sample source image during the training and an operative baseline image and an operative administration image in the medical imaging applications). The encoder comprises 3 groups each of 3 convolutional layers, which groups are followed by corresponding max-pooling layers, and the decoder comprises 3 groups each of 3 convolutional layers, which groups are followed by corresponding up-sampling layers. Each convolutional layer performs a convolution operation through a convolution matrix (filter or kernel) defined by corresponding weights, which convolution operation is performed in succession on limited portions of applied data (receptive field) by shifting the filter across the applied data by a selected number of cells (stride), with the possible addition of cells with zero content around a border of the applied data (padding) to allow applying the filter thereto as well. Batch normalization is then applied (fixing the mean and variance of the corresponding data), followed by an activation function (introducing a non-linearity factor). For example, each convolutional layer applies a filter of 3×3, with a padding of 1 and a stride of 1, with each neuron thereof applying a Rectified Linear Unit (ReLU) activation function. Each max-pooling layer is a pooling layer (down-sampling its applied data), which replaces the values of each limited portion of the applied data (window) with a single value, their maximum in this case, by shifting the window across the applied data by a selected number of cells (stride). For example, each max-pooling layer has a window of 2×2 with a stride of 1. Each up-sampling layer is un-pooling layer (reversing the pooling), which expands each value in a region around it (window), such as using max un-pooling technique (wherein the value is placed in the same position of the maximum used for the down-sampling and it is surrounded by zeros). For example, each up-sampling layer has a window of 2×2. Bypass connections are added between symmetric layers of the encoder and the decoder (to avoid resolution loss) and skip connections are added within each group of convolutional layers and from the input layer to the output layer (to focus on a difference between the input images). The output layer then generates an output image corresponding to the input images (i.e., a sample target image during the training and an operative simulation image in the medical imaging applications), for example, by adding an obtained result (representing the contrast enhancement between the input images increased as required) to the sample/operative baseline image.

The operative neural network 305 reads an operative configurations repository 310 defining one or more (operative) configurations of the operative neural network 310. For example, the operative configurations repository 310 has an entry for each configuration of the operative neural network 305. The entry stores the configuration of the operative neural network 305 (defined by its weights) and the upscaling factor provided by it when operating according to this configuration.

A sample images repository 315 contains information relating to the sample images to be used to train the operative neural network 305. For example, the sample images repository 315 has an entry for each (sample) imaging procedure. The entry stores a plurality of sample images that have been acquired or generated (as described below) for the imaging procedure. Each sample image is defined by a corresponding bitmap, i.e., a matrix of cells (for example, with 512 rows and 512 columns) each containing a value of a basic picture element representing a corresponding location of the body-part of the imaging procedure (for example, a voxel for a basic volume in case of 3D sample images); each voxel value defines a brightness of the voxel (for example, in gray-scale) as a function of a (signal) intensity of a response signal relating to the location. For example, in case of an MRI scanner the response signal represents the response of the location to the magnetic field applied thereto, and in case of a CT scanner the response signal represents the attenuation of the X-ray radiation applied to the location. Moreover, the entry stores one or more acquisition parameters relating to the acquisition of its sample images; particularly, the acquisition parameters comprise one or more extrinsic parameters relating to a setting of the scanner used to acquire the sample images and one or more intrinsic parameters relating to the corresponding body-part (for example, average values for main tissues of the body-part). A collector 320 collects the (acquired) sample images from the central servers, or the control computers, of the lab/health facilities (not shown in the figure). The sample images of at least part of the imaging procedures are incomplete, since they only comprise one or more sample zero-dose images and one or more sample full-dose images of the corresponding body-part. Optionally, the sample images of one or more of the imaging procedures are complete, since they also comprise one or more sample reduced-dose images of the corresponding body-part. The collector 320 writes the sample images repository 315. A pre-processor 325 optionally pre-processes the sample images of each imaging procedure (for example, by co-registering, de-noising and so on); moreover, the pre-processor may also reduce the sample images and/or increase the sample images by generating one or more (generated) sample images (as described in the following). The pre-processor 325 reads/writes the sample images repository 320.

A creator 330 creates a plurality of incomplete sample sets (each formed by at least one sample zero-dose image and at least one sample full-dose image) for each of the imaging procedures providing the incomplete sample images (or at least part thereof) by combining them. The creator 330 reads the sample images repository 320 and it writes an incomplete sample set repository 335 containing information relating to the incomplete sample sets. For example, the incomplete sample sets repository 335 has an entry for each imaging procedure providing the incomplete sample images. The entry stores the corresponding incomplete sample sets, each comprising the bitmaps of its sample zero-dose image(s) and sample full-dose image(s). Moreover, the entry indicates the acquisition parameters of the corresponding imaging procedure (for example, by a pointer thereto in the sample images repository 315).

An analytic engine 340 simulates (or synthesizes) at least one sample reduced-dose image from the zero-dose/full-dose images of each of the incomplete sample sets (or part thereof). The analytic engine 340 exposes a user interface for interacting with it. The analytic engine 340 reads the incomplete sample sets repository 335 and a simulation formulas repository 345, which stores one or more simulation formulas to be used for simulating the sample reduced-dose images.

For example, in case of an MRI scanner, when spin echo is selected as operation mode, the signal intensity defining each voxel value of the sample images (being given by a transverse component of a magnetization of the corresponding location of the body-part during a relaxation of the spins of the protons of the water molecules present therein for returning to their equilibrium condition after application of a magnetic pulse by the RF coil) is expressed by the following signal law:

$$M = {M}_{0} \cdot {e}^{\frac{- T E}{{T}_{2}}} ( 1 - {e}^{\frac{- T R}{{T}_{1}}} ) ,$$

wherein M is the signal intensity, M₀ is a parameter depending on density of the protons, size of the voxel, strength of the magnetic pulse and of the magnetic field, TE is an echo time (between application of the magnetic pulse and receipt of the echo signal), T₂ is a transverse relaxation time of the protons, TR is a repetition time (between successive sequences of magnetic pulses) and T₁ is a longitudinal relaxation time of the protons. In the sample zero-dose images (with no contrast agent), the parameters T₁ and T₂ may be replaced by corresponding diamagnetic values, generally denoted with T₁₀ and T₂₀, respectively, so that the signal intensity (differentiated as M_{zero}) becomes:

$${M}_{z e r o} = {M}_{0} \cdot {e}^{\frac{- T E}{{T}_{20}}} \cdot ( 1 - {e}^{\frac{- T R}{{T}_{10}}} ) .$$

Conversely, when the contrast agent is present in the location the parameters T₁ and T₂ depend on the corresponding diamagnetic values plus corresponding paramagnetic values given by the contrast agent, so that the signal intensity (differentiated as M_{agent}) becomes:

$${M}_{a gent} = {M}_{0} \cdot {e}^{- T E ( \frac{1}{{T}_{20}} + {r}_{2} \cdot c )} \cdot ( 1 - {e}^{- T R ( \frac{1}{{T}_{10}} + {r}_{1} \cdot c )} ) ,$$

wherein r₂ is a transverse relaxivity of the contrast agent, c is a local concentration of the contrast agent in the location and r₁ is a longitudinal relaxivity of the contrast agent. Linearizing this formula as a function of the local concentration of the contrast agent to the first-order approximation of its Taylor series, there is obtained (apart from a negligible error depending on the square of the local concentration of the contrast agent):

$${M}_{a gent} ≅ {M}_{0} \cdot {e}^{\frac{- T E}{{T}_{20}}} \cdot ( 1 - {e}^{\frac{- T R}{{T}_{10}}} ) + F \cdot c = {M}_{z e r o} + F \cdot c ,$$

$$wherein = - \frac{{M}_{0} \cdot {e}^{\frac{- T E}{{T}_{20}}} \cdot ( 1 - {e}^{\frac{- T R}{{T}_{10}}} ) ( {r}_{2} \cdot TE \cdot ( 1 - {e}^{\frac{- T R}{{T}_{10}}} ) + {r}_{1} \cdot TR )}{1 - {e}^{\frac{- T R}{{T}_{10}}}} .$$

Therefore, in the sample full-dose image the signal intensity (differentiated as M_{full}) becomes:

$${M}_{f u l l} ≅ {M}_{z e r o} + F \cdot {c}_{f u l l} ,$$

wherein c_{full} is the local concentration of the contrast agent when administered at the full-dose. Likewise, in the sample reduced-dose image the signal intensity (differentiated as M_{reduced}) becomes:

$${M}_{reduced} ≅ {M}_{zero} + F ⋅ {c}_{reduced} ,$$

wherein c_{reduced} is the local concentration of the contrast agent when administered at the reduced-dose. The local concentration of the contrast agent substantially scales linearly with the amount of contrast agent that is administered, so that:

$${c}_{reduced} = d \cdot {c}_{f ull} ,$$

wherein d is the downscaling factor (corresponding to the upscaling factor k=1/d).
In view of the above, the simulation formula is:

$${M}_{reduced} ≅ {M}_{zero} + F \cdot {c}_{reduced} = {M}_{zero} + F \cdot d \cdot {c}_{full} ;$$

replacing the local concentration at the full-dose c_{full} obtained from the definition of the corresponding intensity signal (M_{full} M_{zero}+F c_{full}), the simulation formula becomes:

$${M}_{reduced} ≅ {M}_{zero} + F \cdot d \cdot \frac{{M}_{full} - {M}_{zero}}{F}$$

$$and then$$

$${M}_{reduced} ≅ {M}_{zero} + d \cdot ( {M}_{full} - {M}_{zero} ) .$$

The same simulation formula is obtained in other operation modes of the MRI scanner, such as gradient echo, MP-RAGE and so on.

Likewise, in case of a CT scanner the signal intensity defining each voxel value of the sample images (given by the X-ray radiation remaining after crossing the corresponding location because of its attenuation) is expressed by the following signal law:

$$I = {I}_{0} \cdot {e}^{- μ \cdot ρ \cdot x} ,$$

wherein I is the signal intensity, I₀ is an initial intensity of the X-ray radiation, μ is a linear attenuation coefficient, ρ is a density and x is a thickness of the location. In the sample zero-dose images (with no contrast agent), the parameters μ, ρ and x are the ones relating to the corresponding material of the body-part, denoted with μ_{M}, μ_{M} and x_{M}, respectively, so that the signal intensity (differentiated as I_{zero}) becomes:

$${I}_{zero} = {I}_{0} \cdot {e}^{- {μ}_{M \cdot } {ρ}_{M} \cdot {x}_{M}} .$$

Conversely, when the contrast agent is present in the location an additional attenuation of the X-ray radiation is caused by it, so that the signal intensity (differentiated as a_{gent}) becomes:

$${I}_{agent} = {I}_{0} \cdot {e}^{- ( {μ}_{M \cdot } {ρ}_{M} \cdot {x}_{M} + {μ}_{A} \cdot {ρ}_{A} \cdot {x}_{A} )} ,$$

wherein μ_{A} is a linear attenuation coefficient, ρ_{A} is the local concentration of the contrast agent and x_{A} is a thickness of the location with this local concentration of the contrast agent. Linearizing this formula as a function of the local concentration of the contrast agent to the first order approximation of its Taylor series, there is obtained (apart from a negligible error depending on the square of the local concentration of the contrast agent):

$${I}_{agent} = {I}_{0} \cdot {e}^{- {μ}_{M \cdot } {ρ}_{M} \cdot {x}_{M}} \cdot {e}^{- {μ}_{A} \cdot ρ \cdot {x}_{A}} = {I}_{zero} \cdot {e}^{- {μ}_{A} \cdot {ρ}_{A} \cdot {x}_{A}} ≅ {I}_{zero} \cdot ( 1 - {μ}_{A} \cdot {ρ}_{A} \cdot {x}_{A} ) .$$

Therefore, in the sample full-dose image the signal intensity (differentiated as I_{full}) becomes:

$${I}_{full} ≅ {I}_{zero} \cdot ( 1 - {μ}_{A} \cdot {ρ}_{full} \cdot {x}_{A} ) ,$$

wherein ρ_{full} is the local concentration of the contrast agent when administered at the full-dose. Likewise, in the sample reduced-dose image the signal intensity (differentiated as I_{reduced}) becomes:

$${I}_{reduced} ≅ {I}_{zero} \cdot ( 1 - {μ}_{A} \cdot {ρ}_{reduced} \cdot {x}_{A} ) ,$$

wherein ρ_{reduced} is the local concentration of the contrast agent when administered at the reduced-dose. The density of the contrast agent substantially scales linearly with the amount of contrast agent that is administered, so that:

$${ρ}_{reduced} = d \cdot {ρ}_{full} ,$$

(d being again the downscaling factor corresponding to the upscaling factor k=1/d). In view of the above, the simulation formula is:

$${I}_{reduced} ≅ {I}_{zero} \cdot ( 1 - {μ}_{A} \cdot {ρ}_{reduced} \cdot {x}_{A} ) = {I}_{zero} \cdot ( 1 - {μ}_{A} \cdot d \cdot {ρ}_{full} \cdot {x}_{A} ) ;$$

replacing the local concentration at the full-dose ρ_{full} obtained from the definition of the corresponding intensity signal (I_{full}≅I_{zero}·(1−μ_{A}·ρ_{full}·x_{A})), the simulation formula becomes:

$${I}_{reduced} ≅ {I}_{zero} \cdot ( 1 - d \cdot ( {μ}_{A} \cdot {x}_{A} \cdot \frac{{I}_{zero} - {I}_{full}}{{μ}_{A} \cdot {x}_{A} \cdot {I}_{zero}} ) )$$

and then as above

$${I}_{reduced} ≅ {I}_{zero} + d \cdot ( {I}_{full} - {I}_{zero} ) .$$

The proposed implementation (wherein the simulation formula is derived from the signal law being linearized with respect to the local concentration of the contrast agent) is computationally very simple, with the loss of accuracy of the sample reduced-dose images so obtained (due to the linearization of the signal law) that is acceptable for the purpose of training the operative neural network.

As an alternative, the signal law is approximated as a function of the local concentration to a higher order of its Taylor series (second, third and so on). In this case, the solution of the obtained equation for the local concentration of the contrast agent at the full-dose provides a corresponding number of values that need to be evaluated to discard any ones of them that are not physically meaningful. This increases the accuracy of the sample reduced-dose images that are simulated (with the higher the order of the approximation the higher the accuracy). As another alternative, the signal law is solved numerically for the local concentration of the contrast agent at the full-dose (again with an evaluation of the possible solutions to discard any ones of them that are not physically meaningful). This further increases the accuracy of the sample reduced-dose images that are simulated.

The analytic engine 340 writes a sample reduced-dose images repository 350 containing the bitmaps of the sample reduced-dose images that have been simulated, each associated with the incomplete sample set used to simulated it in the corresponding repository 335 (for example, by a pointer, a same position and so on). Optionally, a noise corrector 355 corrects the noise of the sample reduced-dose images. The noise corrector 355 reads/writes the sample reduced-dose images repository 350. In fact, the sample zero-dose image and the sample full-dose image of each incomplete sample set contain noise that is propagated to the corresponding sample reduced-dose image according to the simulation formula. However, the noise so obtained (simulated noise) has a statistical distribution that slightly differs from the one of the noise that would have been obtained by actually acquiring the sample reduced-dose image from the corresponding subject to which the contrast agent at the reduced-dose has been administered (real noise). Particularly, the noise of the sample zero-dose image and the noise of the sample full-dose image may be considered to have a normal statistical distribution with zero mean and corresponding standard deviations that propagate to the sample reduced-dose image according to the rules of error (or uncertainty) propagation:

$${σ}_{reduced} = \sqrt{{d}^{2} \cdot {σ}_{full} + {( 1 - d )}^{2} {σ}_{zero}} ,$$

wherein σ_{reduced} is the standard deviation of the noise of the sample reduced-dose image, σ_{full} is the standard deviation of the noise of the sample full-dose image and σ_{zero} is the standard deviation of the noise of the sample zero-dose image (d being again the downscaling factor corresponding to the upscaling factor k=1/d). Assuming that both the noise of the sample zero-dose image and the noise of the sample full-dose image have a same standard deviation σ=σ_{zero}=σ_{full}, in order to make the noise of the sample reduced-dose image having the same normal statistical distribution with zero mean and standard deviation σ, an artificial noise should be injected into the sample reduced-dose image having normal statistical distribution with zero mean and with a standard deviation σ_{artficial} so that:

$${σ}^{2} = {σ}_{reduced}^{2} + {σ}_{artificial}^{2}$$

(in case of injection of the artificial noise in additive form); the standard deviation of the artificial noise is then given by the following noising formula:

$$\begin{matrix}{σ}_{artificial} = \sqrt{{σ}^{2} - {σ}_{reduced}^{2}} = \sqrt{{σ}^{2} - ( {d}^{2} \cdot σ + {( 1 - d )}^{2} \cdot σ )} \\ = σ \cdot \sqrt{2 d ( 1 - d )} .\end{matrix}$$

However, the inventors have found out that better results are obtained by incrementing the (theoretical) value of the standard deviation σ_{artificial} so obtained according to a correction factor being determined empirically (for example, equal to 1.5-2.5, preferably 1.7-2.3 and more preferably 1.9-2.1, such as 2.0).

In case of injection of the artificial noise in multiplicative/convolutional form, the inventors have found out that the standard deviation σ_{artificial} may be set according to the value obtained above for the injection of the artificial noise in additive form, for example, calculated multiplying it by an (empirical) conversion factor equal to 0.05-2.00, preferably 0.1-1.0 and more preferably 0.3-0.7, such as 0.5.

For each of the imaging procedures (or at least part thereof), a combiner 360 generates one or more complete sample sets (each formed by at least one sample zero-dose image, sample full-dose image and sample reduced-dose image). Particularly, when the imaging procedure provides the incomplete sample images, the combiner 360 generates the complete sample sets by combining the incomplete sample sets and the sample reduced-dose images (simulated from the other incomplete sample sets) of the imaging procedure. When the imaging procedure provides the complete sample images, instead, the combiner 360 directly generates the complete sample sets by combining the sample images of the imaging procedure. For this purpose, the combiner 360 reads the incomplete sample sets repository 335 and the reduced dose images repository 350 (for the imaging procedures providing the incomplete sample images) and it reads the sample images repository 315 (for the imaging procedures providing the complete sample images); moreover, the combiner 360 writes a complete sample sets repository 365 containing information relating to the complete sample sets. For example, the complete sample sets repository 365 has an entry for each imaging procedure. The entry stores the corresponding complete sample sets, each comprising the bitmaps of its sample zero-dose image(s), sample full-dose image(s) and sample reduced-dose image(s).

In addition or in alternative, the sample reduced-dose images of the incomplete sample sets are simulated (or synthesized) by an additional (training) machine learning model; for example, the training machine learning model is implemented by a training neural network 370, and particularly an autoencoder convolutional neural network as above (to which reference will be made in the following, with the same considerations that apply to any other implementation thereof). The training neural network 370 is controlled by the analytic engine 340. The training neural network 370 reads a training configuration repository 375, which stores a (training) configuration of the training neural network 370 (i.e., its weights as above). The training neural network 370 reads the incomplete sample sets repository 335 and writes the sample reduced-dose images repository 350.

A training engine 380 trains the operative neural network 305 and the training neural network 370 (when available). The training engine 380 reads the complete sample sets repository 365. The training engine 380 writes the operative configurations repository 310 (of the operative neural network 305); moreover, when the training neural network 370 is available the training engine 380 also writes the training configuration repository 375 (of the training neural network 370), the sample reduced-dose images repository 350 and the complete sample sets repository 365.

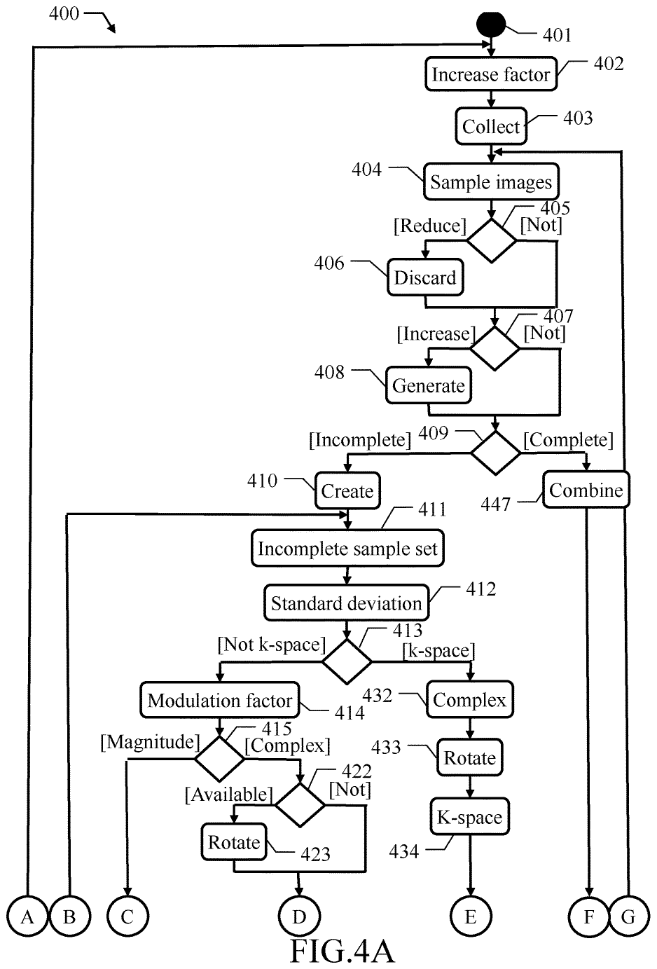

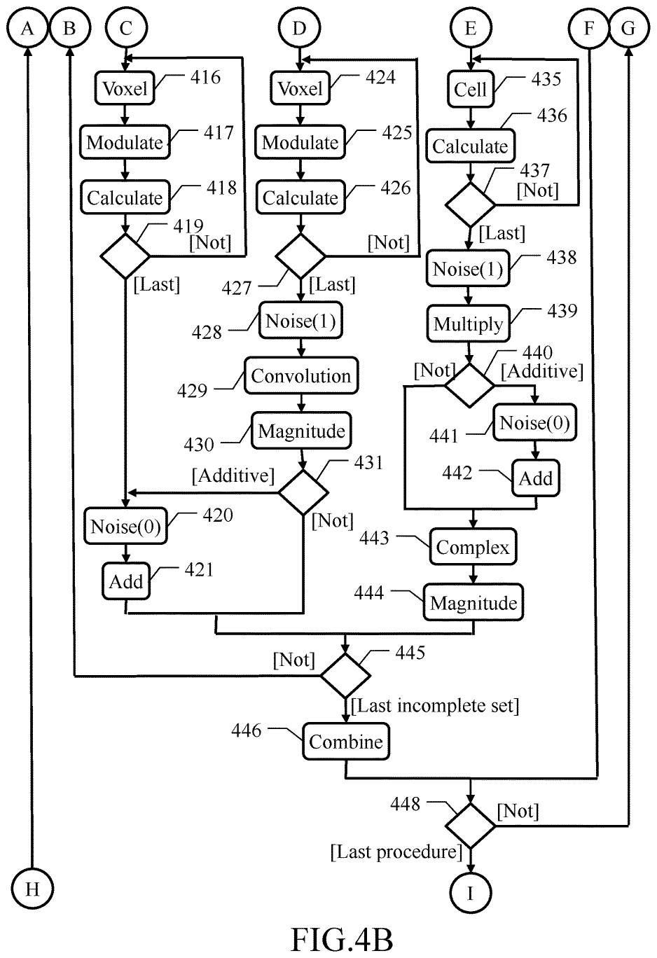

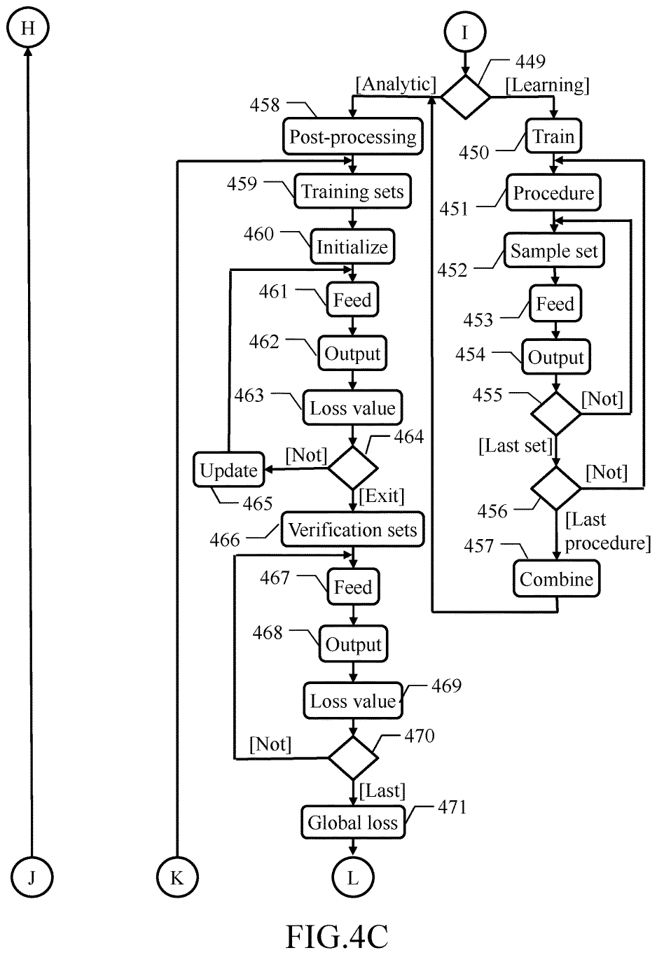

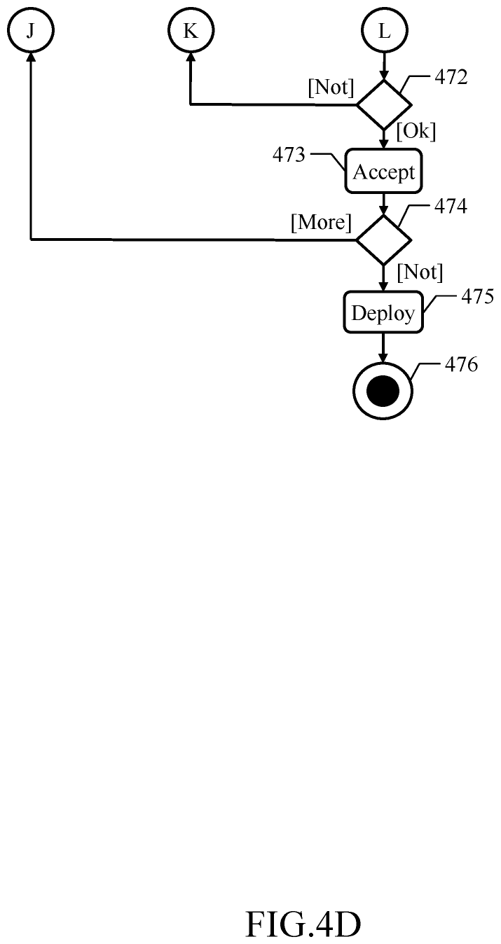

With reference now to FIG. 4A-FIG. 4D, an activity diagram is shown describing the flow of activities relating to an implementation of the solution according to an embodiment of the present disclosure.

Particularly, the diagram represents an exemplary process that may be used to train the operative neural network with a method 400. In this respect, each block may correspond to one or more executable instructions for implementing the specified logical function on the configuration computer.

The process begins at the black start circle 401 whenever the operative neural network needs to be trained. Particularly, this happens before a first delivery of the operative neural network; moreover, this may also happen periodically, in response to any significant change of operative conditions of the imaging systems (for example, delivery of new models of the corresponding scanners, variation of patient population being imaged and so on), in case of a maintenance of the operative neural network or in case of release of a new version of the operative neural network in order to maintain the required performance of the imaging systems or to improve it over time, and so on. In response thereto, the analytic engine at block 402 prompts an operator to enter (via its user interface) an indication of a desired upscaling factor for which the operative neural network has to be trained, also defining the corresponding downscaling factor, for example, as its inverse (unless predefined to a single fixed value).

The collector at block 403 collects the (incomplete and possibly complete) sample images that have been acquired during a plurality of (sample) imaging procedures (saving them in the corresponding repository). The imaging procedures have been performed on body-parts of a same type of the ones for which the operative neural network is intended to be used. The imaging procedures may be either of clinical or pre-clinical type. In fact, the inventors have surprisingly found out that the operative neural network trained with sample images derived (at least in part) from animals nevertheless provides good performance when applied to persons. As a result, the information required to train the operative neural network may be provided in a relatively simple way. In this phase, the pre-processor may also pre-process the sample images (in the corresponding repository). For example, the pre-processor co-registers the sample images to bring them into spatial correspondence (for example, by applying a rigid transformation). In addition or in alternative, the pre-processor de-noises the sample images to reduce their noise. For this purpose, it is possible to use an autoencoder (convolutional neural network). The autoencoder has been trained in an unsupervised way with a plurality of sample images (such as all the ones being collected); particularly, the autoencoder has been trained to optimize its capability of encoding each sample image, ignoring insignificant data thereon (being due to noise) and then decoding the obtained result, so as to reconstruct the same sample image with reduced noise.

The sample images may also comprise corresponding raw-data being used to generate them. For example, in case of an MRI scanner it acquires the raw data as (k-space) images in k-space form. Each k-space image is defined by a matrix of cells with a horizontal axis corresponding to a spatial frequency, or wavenumber k (cycles per unit distance), and a vertical axis corresponding to a phase of the response signals being detected; each cell contains a complex number defining different amplitude components of the corresponding response signal. The k-space image is converted into a corresponding (complex) image in complex form by applying an inverse Fourier transform thereto. The complex image is defined by a matrix of cells for the corresponding voxels; each cell contains a complex number representing the response signal being received from the corresponding location. In the end, the complex image is converted into a corresponding sample image in magnitude form, by setting each voxel value thereof to the modulus of the corresponding complex number in the complex image.

A loop is then entered at block 404, wherein the pre-processor takes the sample images of a (current) imaging procedure into account (starting from a first one in any arbitrary order from the corresponding repository). The flow of activity branches at block 405 according to whether a need exists of reducing the number of the sample images. For example, this operation may be performed to ensure a minimum level of quality of the sample images, to select a single sample zero-dose image, to equalize the number of the sample zero-dose images and the number of the sample full-dose images (and possibly of the sample reduced-dose images), such as by decreasing the sample zero-dose images with respect to the sample full-dose/reduced dose images, and so on. If so, the analytic engine at block 406 discards one or more of the sample images (by deleting them from the corresponding repository). For example, a quality indicator is calculated of each sample image (such as its signal-to-noise ratio); it is then possible to discard all the sample images whose quality indicator is (possibly strictly) lower than an acceptable value, all the sample zero-dose images apart from the one having the highest quality indicator, a number of the sample zero-dose images exceeding the number of the sample full-dose/reduced-dose images with the lower quality indicator and so on. The process descends into block 407 from block 406 or directly from block 405 if no need exists of reducing the number of the sample images.

At this point, the flow of activity branches according to whether a need exists of increasing the number of the sample images. For example, this operation may be performed to ensure a minimum number of the sample images, to have multiple sample full-dose images (such as in clinical imaging procedures wherein only a single sample full-dose image may be available), to equalize the number of the sample zero-dose images and the number of the sample full-dose images (and possibly of the sample reduced-dose images), such as by increasing the sample full-dose/reduced-dose images with respect to the sample zero-dose images, and so on. If so, the pre-processor at block 408 generates one or more (generated) sample images and adds them to the corresponding repository. For example, with reference to the sample full-dose images (similar considerations apply to the sample zero-dose images and to the sample reduced-dose images), it is possible to generate each sample full-dose image by linearly combining two or more (acquired) sample full-dose images, by using an autoencoder being supplied with a corresponding (acquired) sample full-dose image, by adding random noise to a corresponding (acquired) sample full-dose image, and so on. The process descends into block 409 from block 408 or directly from block 407 if no need exists of increasing the number of the sample images.

The flow of activity now branches according to the type of the (possibly reduced/increased) sample images of the imaging procedure. Particularly, blocks 410-446 are executed when the sample images are incomplete, whereas block 447 is executed when the sample images are complete. In both cases, the process then passes to block 448.

With reference in particular to block 410 (incomplete sample images), the creator creates two or more incomplete sample sets for the imaging procedure (each formed by at least one sample zero-dose image and at least one sample full-dose image extracted from the sample images repository) and saves them into the corresponding repository (with a link to its acquisition parameters in the sample images repository). For example, the creator creates the incomplete sample sets by combining a single sample zero-dose image with each of a plurality of sample full-dose images, a same number of sample zero-dose images and sample full-dose images biunivocally, the sample zero-dose images and the sample full-dose image in any possible way, and so on.

A loop is then entered at block 411, wherein the analytic engine takes a (current) incomplete sample set of the imaging procedure into account (starting from a first one in any arbitrary order from the corresponding repository). If necessary, the noise corrector at block 412 calculates the noise of the sample zero-dose image (as a difference between it as acquired and as denoised) and then its (zero-dose) standard deviation; likewise, the noise corrector calculates the noise of the sample full-dose image (as a difference between it as acquired and as denoised) and then its (full-dose) standard deviation. In both cases, the sample (zero-dose/full-dose) images may be denoised with an autoencoder as above. The noise corrector determines a reference standard deviation, for example, equal to an average of the zero-dose standard deviation and the full-dose standard deviation. The noise corrector calculates the standard deviation of the artificial noise to be injected into the corresponding sample source image in additive form (for example, by applying the noising formula to the reference standard deviation and then increasing the obtained result by the correction factor) and/or in multiplicative/convolutional form (for example, multiplying this value by the conversion factor).

The flow of activity branches at block 413 according to a configuration of the analytic engine (for example, selected manually by the operator via its user interface, defined by default or the only one available). Particularly, if the analytic engine is not configured to operate in the k-space, then blocks 414-431 are executed. Otherwise blocks 432-444 are executed. In both cases, the flow of activity merges again at block 445.

With reference in particular to block 414 (not k-space), optionally the analytic engine calculates a modulation factor for modulating the downscaling factor to be used to apply the simulation formula being retrieved from the corresponding repository (for example, selected manually by the operator via its user interface, defined by default or the only one available). In fact, the simulation formula may introduce an approximation, with the higher the local concentration of the contrast agent the higher the approximation. Particularly, starting from a simulation value of the signal intensity (given by the simulation formula) being substantially equal to a real value of the signal intensity (which would have been obtained by actually acquiring the sample reduced-dose image from the corresponding body-part of the subject to which the contrast agent has been administered at the sample reduced-dose) when no contrast agent is present, the simulation value becomes lower and lower than the real value as the local concentration of the contrast agent increases. In order to compensate this loss of the simulation value with respect to the real value, it is possible to increment the value of the downscaling factor being used in the simulation formula (i.e., decrementing its denominator), so as to limit the reduction of the simulation value with respect to the corresponding administration value. More specifically, by solving an equation setting a ratio between the signal law and its approximation for the downscaling factor equal to one, it is obtained that the value of the downscaling factor should be incremented linearly as a function of the local concentration of the contrast agent according to a proportionality factor (modulation factor) depending on the acquisition parameters; the modulation factor is given by a correction formula determined analytically as a function of the acquisition parameters or by values corresponding to the acquisition parameters determined empirically. Therefore, the analytic engine retrieves the acquisition parameters of the incomplete sample set from the incomplete sample sets repository (linking to them in the sample images repository), and then it calculates the modulation factor by applying the correction formula to the acquisition parameters or by retrieving its value corresponding to the acquisition parameters from a pre-defined table.

The flow of activity further branches at block 415 according to the configuration of the analytic engine. Particularly, if the analytic engine is configured to operate on sample images in magnitude form, a loop is entered at block 416 wherein the analytic engine takes a (current) voxel of the sample full-dose image into account (starting from a first one in any arbitrary order). The analytic engine at block 417 modulates the downscaling factor to be used to apply the simulation formula for the voxel. For this purpose, the analytic engine calculates the contrast enhancement of the voxel as a difference between the voxel value of the sample full-dose image and the voxel value of the sample zero-dose image, and then the modulated value of the downscaling factor by multiplying it by the product between the modulation factor and the contrast enhancement. The analytic engine at block 418 calculates the voxel value of the reduced-dose image by applying the simulation formula with the (modulated) downscaling factor to the voxel value of the sample zero-dose image and the voxel value of the sample full-dose image; therefore, in the example at issue the analytic engine subtracts the voxel value of the sample zero-dose image from the voxel value of the sample full-dose image, multiplies this difference by the downscaling factor and adds the obtained result to the voxel value of the sample zero-dose image. The analytic engine then adds the voxel value so obtained to the sample reduced-dose image under construction in the corresponding repository. The analytic engine at block 419 verifies whether a last voxel has been processed. If not, the flow of activity returns to block 416 to repeat the same operations on a next voxel. Conversely (once all the voxels have been processed) the corresponding loop is exited by descending into block 420.

At this point, the noise corrector injects the artificial noise into the sample reduced-dose image so obtained in additive form. For this purpose, the noise corrector generates the artificial noise as a (noise) matrix of cells having the same size as the sample reduced-dose image; the noise matrix contains random values having normal statistical distribution with zero mean and standard deviation equal to the one of the artificial noise. The noise corrector at block 421 adds the noise matrix to the sample reduced-dose image voxel-by-voxel in the corresponding repository. The process then continues to block 445.

With reference back to block 415, if the analytic engine is configured to operate on sample images in complex form, the flow of activity branches at block 422 according to their availability. If the sample zero-dose image and the sample full-dose image are already available in complex form, the analytic engine at block 423 performs a phase correction by rotating a vector representing the complex number of each cell thereof so as to cancel its argument (maintaining the same modulus). This operation allows obtaining the same result of the application of the simulation formula even when operating on the sample zero-dose image and sample full-dose image in complex form (since all the operations applied to the corresponding complex numbers without imaginary part are equivalent to apply them to the corresponding modulus). The process then continues to block 424. The same point is also reached directly from block 422 if the sample zero-dose image and the sample full-dose image are available in magnitude form; in this case, the sample zero-dose image and the sample full-dose image are considered directly as in complex form, with each voxel value thereof (real number) being a complex number with imaginary part equal to zero.

Similar operations as above are now performed for generating the sample reduced-dose image from the sample zero dose-image and the sample full-dose image working on them in complex form. Particularly, a loop is entered wherein the analytic engine takes a (current) voxel of the sample full-dose image into account (starting from a first one in any arbitrary order). The analytic engine at block 425 modulates the downscaling factor by calculating the contrast enhancement of the voxel (as the different between the modulus of the voxel value of the sample full-dose image and the modulus of the voxel value of the sample zero-dose image) and then the modulated value of the downscaling factor by multiplying it by the product between the modulation factor and the contrast enhancement. The analytic engine at block 426 calculates the voxel value of the sample reduced-dose image by applying the simulation formula with the (modulated) downscaling factor to the voxel value of the sample zero-dose image and the voxel value of the sample full-dose image; the analytic engine then adds the voxel value so obtained to the sample reduced-dose image under construction in the corresponding repository. The analytic engine at block 427 verifies whether a last voxel has been processed. If not, the flow of activity returns to block 424 to repeat the same operations on a next voxel. Conversely (once all the voxels have been processed) the corresponding loop is exited by descending into block 428.

At this point, the noise corrector injects the artificial noise into the sample reduced-dose image so obtained in convolutional form. For this purpose, the noise corrector generates the artificial noise as a (noise) matrix of cells having the same size as the sample reduced-dose image; the noise matrix contains random complex values having normal statistical distribution with unitary mean and standard deviation equal to the one of the artificial noise. The noise corrector at block 429 then performs a convolution operation on the sample reduced-dose image in the corresponding repository through the noise matrix (for example, by shifting the noise matrix across the sample reduced-dose image by a single stride in a circular way, wrapping around the sample reduced-dose image in every direction). The analytic engine at block 430 converts the sample reduced-dose image so obtained into magnitude form; for this purpose, the analytic engine replaces each voxel value of the sample reduced-dose image (now generally a complex number) with its modulus. The flow of activity further branches at block 431 according to the configuration of the analytic engine. Particularly, if the analytic engine is configured to inject the artificial noise into the sample reduced-dose image in additive form as well, the process continues to block 420 for performing the same operations described above (then descending into block 445). Conversely, the process descends into block 445 directly.

With reference instead to block 432 (k-space), the analytic engine takes the sample zero-dose image and the sample full-dose image in complex form into account (directly if available or by converting them from k-space form by applying the inverse Fourier transform thereto). As above, the analytic engine at block 433 performs a phase correction by rotating the vector representing the complex number of each cell of the sample zero-dose image and the sample full-dose image in complex form so as to cancel its argument (maintaining the same modulus). The analytic engine at block 434 converts the sample zero-dose image and the sample full-dose image from complex form into k-space form by applying a Fourier transform thereto.

The sample reduced-dose image is now generated from the sample zero dose-image and the sample full-dose image working on them in k-space form. Particularly, a loop is entered at block 435 wherein the analytic engine takes a (current) cell of the sample full-dose image into account (starting from a first one in any arbitrary order). The analytic engine at block 436 calculates the cell value of the sample reduced-dose image by applying the simulation formula with the (original) downscaling factor to the cell value of the sample zero-dose image and the cell value of the sample full-dose image; the analytic engine then adds the cell value so obtained to the sample reduced-dose image under construction in the corresponding repository. The analytic engine at block 437 verifies whether a last cell has been processed. If not, the flow of activity returns to block 435 to repeat the same operations on a next cell. Conversely (once all the cells have been processed) the corresponding loop is exited by descending into block 438.

At this point, the noise corrector injects the artificial noise into the sample reduced-dose image so obtained in multiplicative form. For this purpose, the noise corrector generates the artificial noise as a (noise) matrix of cells having the same size as the sample reduced-dose image; the noise matrix contains random complex values having normal statistical distribution with unitary mean and standard deviation equal to the one of the artificial noise. The noise corrector at block 439 multiplies the sample reduced-dose image by the noise matrix cell-by-cell in the corresponding repository. The flow of activity further branches at block 440 according to the configuration of the analytic engine. Particularly, if the analytic engine is configured to inject the artificial noise into the sample reduced-dose image in additive form as well, the process continues to block 441, wherein the noise corrector generates the artificial noise as a (further) noise matrix of cells (having the same size as the sample reduced-dose image) now containing random complex values having normal statistical distribution with null mean and standard deviation equal to the one of the artificial noise. The noise corrector at block 442 adds the noise matrix to the sample reduced-dose image cell-by-cell in the corresponding repository. The process then continues to block 443; the same point is also reached directly from block 440 if the analytic engine is not configured to inject the artificial noise into the sample reduced-dose image in additive form. At this point, the analytic engine converts the sample reduced-dose image from k-space form into complex form by applying the inverse Fourier transform thereto. The analytic engine at block 444 converts the sample reduced-dose image from complex form into magnitude form by replacing each voxel value thereof with its modulus. The process then descends into block 445. Alternatively, not shown in the figure, the artificial noise may be injected in additive form (when it is required) into the sample reduced-dose image in magnitude form by continuing from block 444 to block 420 for performing the same operations described above (then descending into block 445).

With reference now to block 445, the analytic engine verifies whether a last incomplete sample set of the imaging procedure has been processed. If not, the flow of activity returns to block 411 to repeat the same operations on a next incomplete sample set. Conversely (once all the incomplete sample sets, or a selected part thereof, have been processed) the corresponding loop is exited by descending into block 446.

At this point, the combiner generates one or more complete sample sets (each formed by at least one sample zero-dose image, sample full-dose image and sample reduced-dose image) for the imaging procedure (and saves them into the corresponding repository), by combining the incomplete sample sets with the sample reduced-dose images being generated from the other incomplete sample sets (retrieved from the corresponding repositories). This result may be achieved in different ways. Particularly, in an embodiment a half of the incomplete sample sets (in this case, without the need of simulating the corresponding sample reduced-dose images) and the sample reduced-dose images being simulated from another half of the incomplete sample sets are combined biunivocally. Therefore, if the incomplete sample sets are N, this provides INT(N/2) complete sample sets. For example, a scenario is considered with four incomplete sample sets B1-F1, B2-F2, B3-F3, B4-F4 (formed by corresponding sample zero-dose images B1, B2, B3, B4 and sample full-dose images F1, F2, F3, F4) and four sample reduced-dose images R1, R2, R3, R4 that may be simulated from them. If the incomplete sample sets B1-F1, B2-F2 and the sample reduced-dose images R3, R4 are taken into account, the complete sample sets (INT(4/2)=2) may be B1-F1-R3, B2-F2-R4. This embodiment provides high diversity of the complete sample sets that will be used to train the operative neural network. In another embodiment, the incomplete sample sets and the sample reduced-dose images being simulated from the other incomplete sample sets are combined in all possible ways. Therefore, if the incomplete sample sets and the corresponding sample reduced-dose images are N, this provides N·(N−1) complete sample sets. Considering the same example of above, the complete sample sets (4·3=12) will be B1-F1-R2, B1-F1-R3, B1-F1-R4, B2-F2-R3, B2-F2-R4, B2-F2-R1, B3-F3-R4, B3-F3-R1, B3-F3-R2, B4-F4-R1, B4-F4-R2, B4-F4-R3. This implementation provides a high amount of complete sample sets that will be used to train the operative neural network. In still another embodiment, the incomplete sample sets and the sample reduced-dose images being simulated from the other incomplete sample sets are combined biunivocally. Therefore, if the incomplete sample sets and the corresponding sample reduced-dose images are N, this provides N complete sample sets. Considering the same example of above, the complete sample sets (4) may be B1-F1-R2, B2-F2-R3, B3-F3-R4, B4-F4-R1. This embodiment provides a compromise between high diversity and high amount of the complete sample sets that will be used to train the operative neural network. The process then descends into block 448.

With reference now to block 447 (complete sample images), the combiner directly generates one or more complete sample sets (each formed by at least one sample zero-dose image, sample full-dose image and sample reduced-dose image) for the imaging procedure (and saves them into the corresponding repository), by combining its sample images (retrieved from the corresponding repository). This result may again be achieved in different ways. Particularly, it is possible to combine a same number of sample full-dose images and sample reduced-dose images biunivocally and then each pair thereof so obtained with a same (single) sample zero-dose image, a same number of sample zero-dose images, sample full-dose images and sample reduced-dose images biunivocally, the sample full-dose images and the sample reduced-dose images in all possible ways and then each pair thereof so obtained with a same (single) sample zero-dose image, the sample zero-dose images, the sample full-dose images and the sample reduced-dose images in all possible ways, and so on. The process then descends into block 448.

With reference now to block 448, there is verified whether a last imaging procedure has been processed. If not, the flow of activity returns to block 404 to repeat the same operations on a next imaging procedure. Conversely (once all the imaging procedures have been processed) the corresponding loop is exited by descending into block 449. As a result, the complete sample sets repository may store a mix of (simulated) complete sample sets whose sample reduced-dose images have been simulated and (acquired) complete sample sets whose sample reduced-dose images have been acquired; for example, the acquired complete sample sets are 1-20%, preferably 5-15% and still more preferably 6-12%, such as 10% of a total number of the (simulated/acquired) complete sample sets. This may further increase the quality of the training of the operative neural network with a limited additional effort (especially when the acquired complete sample sets are obtained from pre-clinical imaging procedures).

With reference now to block 449, the flow of activity branches according to an operative mode of the configuration computer. If the training neural network is available to simulate the sample reduced-dose images (to be used to train the operative neural network), the training engine at block 450 trains it by using the complete sample sets (retrieved from the corresponding repository). For example, the same operations described below for training the operative neural network may be performed, with the difference that the training neural network is now optimized to generate the sample reduced-dose images from the corresponding sample zero-dose images and sample full-dose images; in this case, it is also possible to use a more complex loss function to improve performance of the training neural network, for example, with an approach making use of Generative Adversarial Networks (GANs). The training engine saves the configuration of the training neural network so obtained into the corresponding repository; at the same time, the training engine deletes the sample reduced-dose images that have been simulated analytically and the corresponding complete sample sets from their repositories. A loop is then entered at block 451 for simulating a refined version of the sample reduced-dose images for the imaging procedures providing the incomplete sample images. For this purpose, the analytic engine takes a (current) imaging procedures providing the incomplete sample images into account (starting from a first one in any arbitrary order in the sample images repository). The analytic engine at block 452 then takes a (current) incomplete sample set of the imaging procedure into account (starting from a first one in any arbitrary order from the corresponding repository). The analytic engine at block 453 feeds the sample zero-dose image and the sample full-dose image of the incomplete sample set to the training neural network. Moving to block 454, the training neural network outputs the corresponding sample reduced-dose image, which is saved into the corresponding repository. The analytic engine at block 455 verifies whether a last incomplete sample set has been processed. If not, the flow of activity returns to block 452 to repeat the same operations on a next incomplete sample set. Conversely (once all the incomplete sample sets, or a selected part thereof, have been processed) the corresponding loop is exited by passing to block 456, wherein the analytic engine now verifies whether a last imaging procedure has been processed. If not, the flow of activity returns to block 451 to repeat the same operations on a next imaging procedure. Conversely (once all the imaging procedures have been processed) the corresponding loop is exited by descending to block 457. At this point, the combiner generates one or more complete sample sets for the imaging procedure (and saves them into the corresponding repository), by combining the incomplete sample sets with the sample reduced-dose images being generated from the other incomplete sample sets (retrieved from the corresponding repositories) as above.

The process then passes to block 458 for training the operative neural network with the completed sample sets so obtained. This implementation improves the accuracy of the sample reduced-dose images and then the performance of the operative neural network being trained with the corresponding complete sample sets. The same point is also reached directly from block 449 if the complete sample sets generated by the analytic engine are to be used directly for training the operative neural network since no training neural network is available. This implementation is particularly simple and fast; at the same time, the accuracy of the sample reduced-dose images being simulated analytically is sufficient for the purpose of training the operative neural network with acceptable performance.

In both cases, the training engine now performs this operation, in order to find optimized values of the weights of the operative neural network that optimize its performance. First of all, optionally the training engine may post-process the sample images of each complete sample set. For example, the training engine normalizes the sample images by scaling their voxel values to a (common) pre-defined range. Moreover, the training engine performs a data augmentation procedure by generating (new) complete sample sets from each (original) complete sample set, so as to reduce overfitting in the training of the operative neural network. For example, the new complete sample sets are generated by rotating the sample images of the original complete sample set, such as incrementally by 1-5° from 0° to 90°, and/or by flipping them horizontally/vertically. In any case, the training engine at block 459 selects a plurality of training sets by sampling the complete sample sets in the corresponding repository to a percentage thereof (for example, 50% selected randomly). The training engine at block 460 initializes the weights of the operative neural network randomly. A loop is then entered at block 461, wherein the training engine feeds the sample zero-dose image and the sample reduced-dose image of each training set to the operative neural network. In response thereto, the operative neural network at block 462 outputs a corresponding output image, which should be equal to the sample full-dose image (ground truth) of the training set. The training engine at block 463 calculates a loss value based on a difference between the output image and the sample full-dose image; for example, the loss value is given by the Mean Absolute Error (MAE) calculated as the average of the absolute differences between the corresponding voxel values of the output image and of the sample full-dose image. The training engine at block 464 verifies whether the loss value is not acceptable and it is still improving significantly. This operation may be performed either in an iterative mode (after processing each training set for its loss value) or in a batch mode (after processing all the training sets for a cumulative value of their loss values, such as an average thereof). If so, the training engine at block 465 updates the weights of the operative neural network in an attempt to improve its performance. For example, the Stochastic Gradient Descent (SGD) algorithm, such as based on the ADAM method, is applied (wherein a direction and an amount of the change is determined by a gradient of a loss function, giving the loss value as a function of the weights being approximated with a backpropagation algorithm, according to a pre-defined learning rate). The process then returns to block 461 to repeat the same operations. With reference again to block 464, if the loss value has become acceptable or the change of the weights does not provide any significant improvement (meaning that a minimum, at least local, or a flat region of the loss function has been found) the loop is exited. The above-described loop is repeated a number of times (epochs), for example, 100-300, by adding a random noise to the weights and/or starting from different initializations of the operative neural network to find different (and possibly better) local minimums and to discriminate the flat regions of the loss function.

Once a configuration of the operative neural network has been found providing an optimal minimum of the loss function, the process continues to block 466 wherein the training engine performs a verification of the performance of the operative neural network so obtained. For this purpose, the training engine selects a plurality of verification sets from the sample sets in the corresponding repository (for example, the ones different from the training sets). A loop is then entered at block 467, wherein the training engine feeds the sample zero-dose image and the sample reduced-dose image of a (current) verification set (starting from a first one in any arbitrary order) to the operative neural network. In response thereto, the operative neural network at block 468 outputs a corresponding output image, which should be equal to the sample full-dose image of the verification set. The training engine at block 469 calculates the loss value as above based on the difference between the output image and the sample full-dose image. The training engine at block 470 verifies whether a last verification set has been processed. If not, the flow of activity returns to block 467 to repeat the same operations on a next verification set. Conversely (once all the verification sets have been processed) the loop is exited by descending into block 471. At this point, the training engine determines a global loss of the above-mentioned verification (for example, equal to an average of the loss values of all the verification sets). The flow of activity branches at block 472 according to the global loss. If the global loss is (possibly strictly) higher than an acceptable value, this means that the capability of generalization of the operative neural network (from its configuration learned from the training sets to the verification sets) is too poor; in this case, the process returns to block 459 to repeat the same operations with different training sets and/or training parameters (such as learning rate, epochs and so on). Conversely, if the global loss is (possibly strictly) lower than the acceptable value, this means that the capability of generalization of the operative neural network is satisfactory; in this case, the training engine at block 473 accepts the configuration of the operative neural network so obtained, and saves it into the corresponding repository in association with its value of the upscaling factor.

The analytic engine at block 474 verifies whether the configuration of the operative neural network has been completed. If not, the process returns to block 402 for repeating the same operations in order to configure the operative neural network for a different upscaling factor. Conversely, once the configuration of the operative neural has been completed, the configurations of the operative neural network so obtained are deployed at block 475 to a batch of instances of the control computers of corresponding imaging systems (for example, by preloading them in the factory in case of first delivery of the imaging systems or by uploading them via the network or a removable storage unit in case of upgrade of the imaging systems). The process then ends to the concentric white/black stop circles 476.

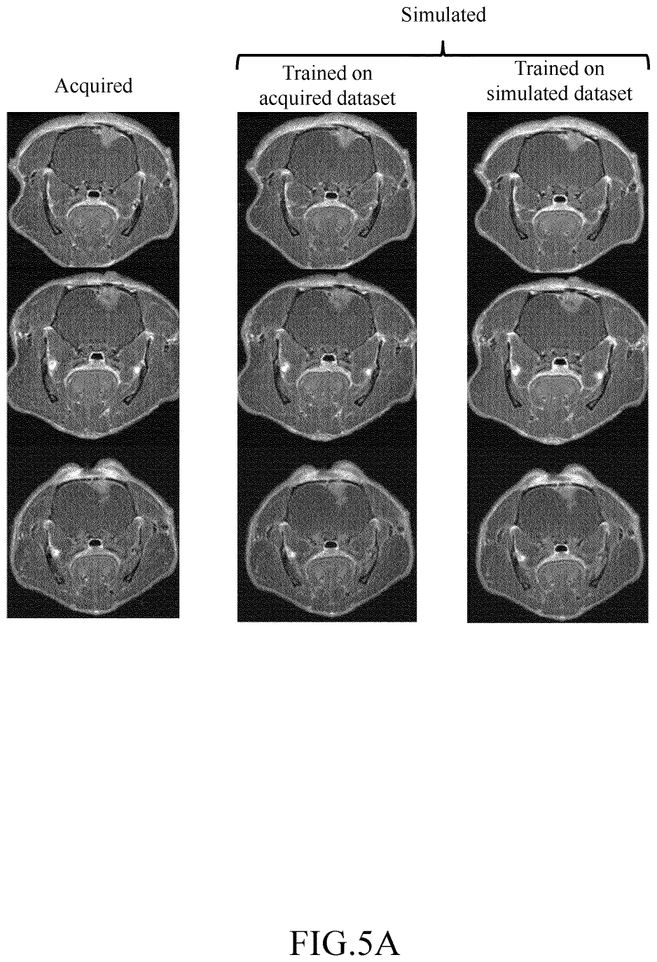

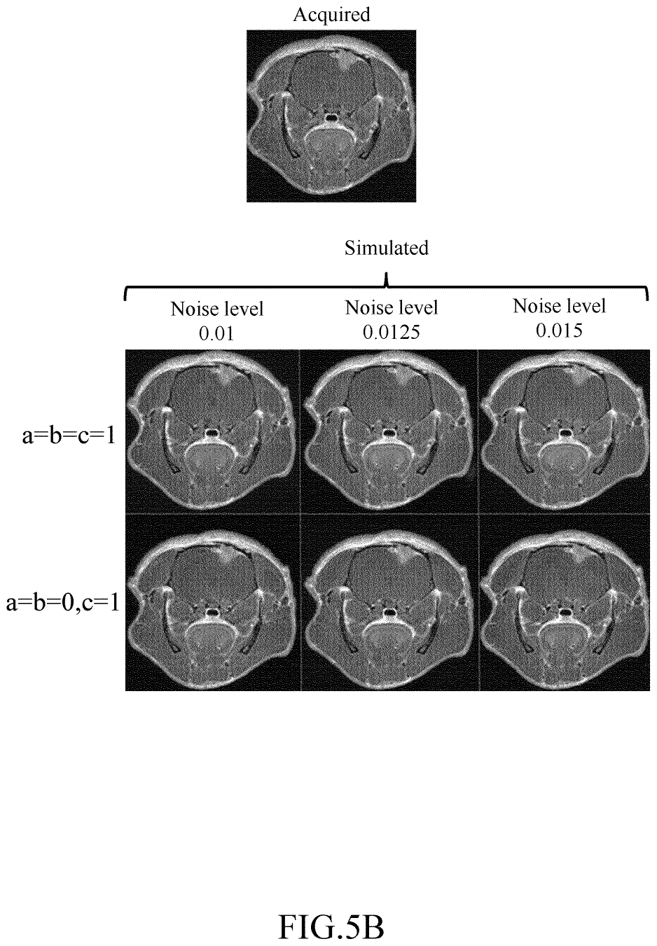

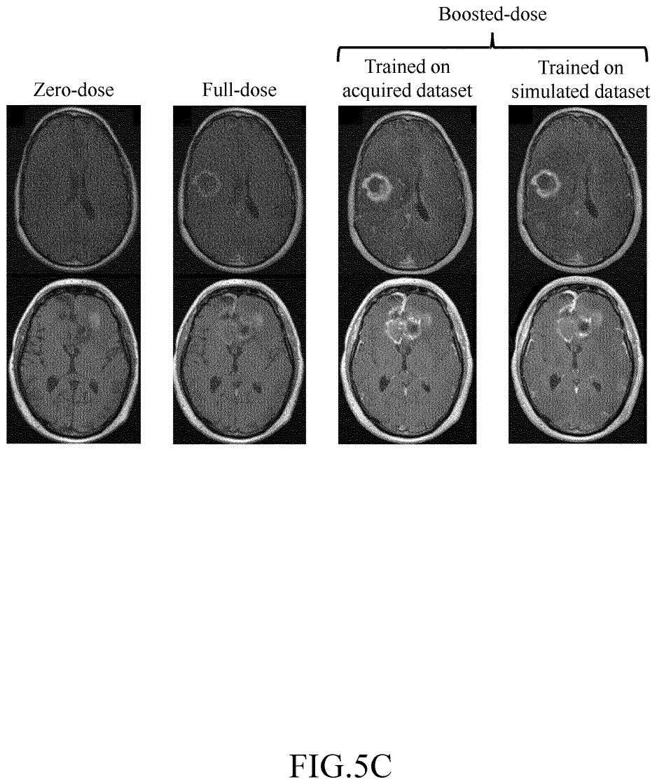

With reference now to FIG. 5A-FIG. 5C, representative examples are shown of experimental results relating to the solution according to an embodiment of the present disclosure.

Particularly, a dedicated pre-clinical study was carried out on rats bearing the following two cerebral lesions: a C6 glioma tumor (n=36 animals) and a cerebral ischemia pathology (n=42 animals). All the animals underwent a surgical procedure for inducing the lesion. Animals that survived the surgical procedure and which, during the two following weeks (i.e., the time window required for a pathology development of the lesion), showed only limited or none clinical signs were enrolled for imaging procedures of MRI type (i.e., typically 2 for each animal, 3 only in limited cases). The imaging procedures were carried out using a Gadolinium based contrast agent and a pre-clinical scanner spectrometer Pharmascan by Bruker Corporation (trademarks thereof), that operates at 7T and is equipped with a rat head volume coil with 2 channels. The CE-MR protocol used during each imaging procedure was the following:
first pre-contrast acquisition of a standard T1-weighted sequence (first sample zero-dose image);
second pre-contrast acquisition of a standard T1-weighted sequence (second sample zero-dose image);
intravenous administration of the contrast agent at a sample reduced-dose equal to 0.01 mmol Gd/kg;
post-contrast acquisition of a T1-weighted sequence (sample reduced-dose image);
further intravenous administration of the contrast agent at 0.04 mmol Gd/kg shortly after the previous one so that they sum into a sample full-dose equal to 0.05 mmol Gd/kg;
first post-contrast acquisition of a T1-weighted sequence (first sample full-dose image);
second post-contrast acquisition of a T1-weighted sequence (second sample full-dose image).

The study led to the acquisition of 130 3D MRI sample images (i.e., 61 on glioma bearing rats and 69 on ischemia bearing rats) each consisting of 24 slices. The acquired sample images were used to build two datasets:
acquired dataset that comprises, for each imaging procedure, the first zero-dose image, the sample reduced-dose image and the first sample full-dose image being all acquired;
simulated dataset that comprises, for each imaging procedure, the first sample zero-dose image and the first sample full-dose image being acquired and a sample reduced-dose image being simulated (with downscaling factor d=1/5) from the second sample zero-dose image and the second sample full-dose image.

Using the sample full-dose images as ground truth, an operative convolutional neural network (as described in “Deep artifact learning for compressed sensing and parallel MRI”, Dongwook Lee, Jaejun Yoo and Jong Chul Ye, available at https://arxiv.org/abs/1703.01120) was trained with the following hyperparameters:
learning rate=0.01
decay=0.001
composite LOSS=mean absolute error (MAE)+mean absolute error in the Fourier transform domain (fftMAE)+perceptual loss (PL) with network VGG19 cropped at layer 4 pre-trained on ImageNET dataset (available at https://www.image-net.org/)
relative weights of composite loss: a=b=c=1 and a=b=0,c=1 (with “a” for MAE, “b” for fftMAE and “c” for PL)
level of noise (o_{artificial}) injected into sample reduced-dose images being simulated: 0.01, 0.0125 and 0.015.

Different instances of the operative neural network were trained with the acquired dataset and with the simulated dataset, using the corresponding full-dose images as ground truth. These instances of the neural network were then applied to the sample reduced-dose images comprised in the acquired dataset (to verify their capability in restoring the effect of the full-dose of the contrast agent) and to operative full-dose images acquired in medical imaging applications with the full-dose of the contrast agent (to verify their capability in boosting the effect of the contrast agent).

Starting from FIG. 5A, three representative examples are shown of a sample full-dose image being acquired and of a corresponding sample full-dose image being simulated by the operative neural network trained on the acquired dataset and on the simulated dataset. As can be seen, the sample full-dose images being simulated are very similar to the sample full-dose images being acquired; this is true for the operative neural network being trained with either the acquired dataset or the simulated dataset. Particularly, the use of the operative neural network being trained with the simulated dataset do not substantially introduce artefacts in the sample full-dose images that are simulated by it.

Moving to FIG. 5B, a representative example is shown of a sample full-dose images being acquired and being simulated (by the operative neural network trained with the simulated dataset) using different hyperparameters (i.e., relative weights a=b=c=1 and a=b=0, c=1, and noise level of 0.01, 0.0125 and 0.015). As can been seen, the quality of the sample full-dose image being simulated may be further improved by tuning the hyperparameters, so as to increase the similarity with the sample full-dose image being acquired under different criteria (such as enhancement of regions perfused with the contrast agent, signal intensity of enhanced and unenhanced regions, and so on).

Moving to FIG. 5C, two representative examples are shown of an operative zero-dose image, an operative full-dose image being acquired during an imaging procedure on a (human) patient and of a corresponding operative boosted-dose image (upscaling factor k=5) being simulated by the operative neural network trained on the acquired dataset and on the simulated dataset (both of them based on imaging procedures on animals). As can be seen, the operative boosted-dose images being simulated by the operative neural network trained with the simulated dataset are superimposable on the operative boosted-dose images being simulated by the operative neural network trained with the acquired dataset; in both cases, the operative boosted-dose images improve the contrast enhancement without substantially introducing artefacts.

### Modifications

In order to satisfy local and specific requirements, a person skilled in the art may apply many logical and/or physical modifications and alterations to the present disclosure. More specifically, although this disclosure has been described with a certain degree of particularity with reference to one or more embodiments thereof, it should be understood that various omissions, substitutions and changes in the form and details as well as other embodiments are possible. Particularly, different embodiments of the present disclosure may be practiced even without the specific details (such as the numerical values) set forth in the preceding description to provide a more thorough understanding thereof, conversely, well-known features may have been omitted or simplified in order not to obscure the description with unnecessary particulars. Moreover, it is expressly intended that the features described in each complete sentence may be implemented independently of the features described in the other sentences (except for those strictly necessary functionally). In any case, specific features described in connection with any embodiment of the present disclosure may be incorporated in any other embodiment as a matter of general design choice. Moreover, items presented in a same group and different embodiments, examples or alternatives are not to be construed as de facto equivalent to each other (but they are separate and autonomous entities). In any case, each numerical value should be read as modified according to applicable tolerances; particularly, unless otherwise indicated, the terms “substantially”, “about”, “approximately” and the like should be understood as within 10%, preferably 5% and still more preferably 1%. Moreover, each range of numerical values should be intended as expressly specifying any possible number along the continuum within the range (comprising its end points). Ordinal or other qualifiers are merely used as labels to distinguish elements with the same name but do not by themselves connote any priority, precedence or order. The terms include, comprise, have, contain, involve and the like should be intended with an open, non-exhaustive meaning (i.e., not limited to the recited items), the terms based on, dependent on, according to, function of and the like should be intended as a non-exclusive relationship (i.e., with possible further variables involved), the term a/an should be intended as one or more items (unless expressly indicated otherwise), and the term means for (or any means-plus-function formulation) should be intended as any structure adapted or configured for carrying out the relevant function.

For example, an embodiment provides a method for training a machine learning model. However, the machine learning model may be of any type (for example, a dense neural network, a convolutional neural network, a generative adversarial network, a linear and not linear regression model, such as a polynomial regression, support vector regression, nearest neighbors regression, gaussian process regression and the like, a probabilistic model, such as a Bayesian network, Markov random field and the like, used in combination with optimization methods, when required, such as those based on gradient descent and other more sophisticated methods such as genetic algorithms, and so on).

In an embodiment, the machine learning model is for use in medical imaging applications. However, the medical imaging applications may be of any type (for example, diagnostic, therapeutic or surgical applications, based on MRI, CT, fluoroscopy, fluorescence or ultrasound techniques, and so on).

In an embodiment, the method comprises the following steps under the control of a computing system. However, the computing system may be of any type (see below).

In an embodiment, the method comprises providing (to the computing system) a plurality of incomplete sample sets for each of a plurality of imaging procedures. However, the imaging procedures may be in any number and of any type (for example, pre-clinical, clinical and so on), and the incomplete sample sets of each imaging procedure may be in any number (either the same or different among the imaging procedures); moreover, the incomplete sample sets may be provided in any way (for example, already formed or created from corresponding sample images, collected from any sources, such as health/lab facilities, in any way, such downloaded over the Internet from the central servers of the facilities wherein they have been gatherer from the corresponding imaging systems either automatically over the corresponding LANs or manually by means of removable storage units, loaded manually from removable storage units wherein they have been copied from the central servers or from (stand-alone) imaging systems, and so on). In any case, this is a (computer-implemented) data-processing method that is performed independently of the acquisition of the sample images (without requiring any interaction with the corresponding subjects).

In an embodiment, the imaging procedures relate to corresponding body-parts of subjects. However, the body-parts may be in any number, of any type (for example, organs, regions thereof, tissues, bones, joints and the like, either the same as or different from the one for which the machine learning model is to be used in the medical imaging procedures, and so on) and in any condition (for example, healthy, pathological with any lesions and so on); moreover, the body-parts may belong to any number and type of subjects (for example, animals, persons and so on).

In an embodiment, each of the incomplete sample sets comprises at least one sample target image. However, the sample target images may be in any number (for example, axial, coronal and/or sagittal images, corresponding to different doses of the contrast agent and so on) and of any type (for example, in any form such as magnitude, complex, k-space and the like, with any size, resolution, chromaticity, bit depth and the like, relating to any locations of the body-parts, such as voxels for 3D images, pixels for 2D images and so on).

In an embodiment, the sample target image is representative of the corresponding body-part of the subject to which a contrast agent has been administered at a sample target-dose. However, the contrast agent may be of any type (for example, any targeted contrast agent, such as based on specific or non-specific interactions, any non-targeted contrast agent, and so on) and it may have been administered in any way ensuring that is has perfused the body-part (for example, with any advance with respect to the imaging procedure, down to immediately before it, intravenously, intramuscularly, orally and so on) at any sample target-dose (for example, equal to the full-dose, lower or higher than the full-dose, and so on).

In an embodiment, each of the incomplete sample sets comprises at least one sample baseline image. However, the sample baseline images may be in any number (for example, axial, coronal and/or sagittal images, corresponding to zero and/or different doses of the contrast agent, and so no) and of any type (for example, either the same or different with respect to the sample target images).

In an embodiment, the sample baseline image is representative of the corresponding body-part of the subject without the contrast agent. However, the sample baseline image may have been acquired in any way ensuring that no contrast agent significantly affects its content (for example, preceding the administration of the contrast agent by any advance, following a possible previous administration of the contrast agent by any delay and so on).

In an embodiment, the sample baseline image is representative of the corresponding body-part of the subject to which the contrast agent has been administered at a sample baseline-dose lower than the sample target-dose. However, the sample baseline-dose may have any value (for example, either lower or higher than the sample source dose).

In an embodiment, the method comprises simulating (by the computing system) a plurality of sample source images, at least one of the sample source images being simulated from each of at least part of the incomplete sample sets of each of the imaging procedures. However, the sample source images may be simulated from any number of the incomplete sample sets (up to all of them), in any number from each of them (for example, for different values of the downscaling factor) and in any way (for example, operating in any domain, such as magnitude, complex, k-space and the like, with or without any pre-processing, such as registration, normalization, denoising, such as with an autoencoder, analytic techniques based on block-matching, shrinkage fields, Wavelet transform, smoothing filters and the like, distortion correction, filtering of abnormal sample images and the like, with or without any post-processing, such as any registration, normalization, noise injection and so on). For example, in an embodiment the sample source images are generated analytically (such as by applying the simulation formula in case of single sample baseline/target images, by interpolation in case of multiple sample baseline/target images and so on). In another embodiment, a preliminary version of the sample source images is generated analytically, a further machine learning model is trained with a preliminary version of complete sample sets based on the sample source images generated analytically and then a refined version of the sample source images is generated by the further machine learning model being trained. In still another embodiment, the sample source images are generated by a further machine learning model being trained with further sample sets acquired independently.

In an embodiment, the sample source image is simulated to be representative of the corresponding body-part mimicking administration to the corresponding subject of the contrast agent at a sample source-dose lower than the sample target-dose (with a ratio between the sample source-dose and the sample target-dose equal to a downscaling factor). However, the sample source-dose may have any value, either in absolute or relative terms (for example, with the sample source-dose lower than, equal to or higher than the full-dose, with the sample source-dose and the sample target-dose defining any downscaling factor, and so on).

In an embodiment, the method comprises generating (by the computing system) one or more complete sample sets for each of the imaging procedures. However, the complete sample sets of each imaging procedure may be in any number.

In an embodiment, each of the complete sample sets comprises the sample baseline image and the sample target image of one of the incomplete sample sets of the imaging procedure and the sample source image being simulated from another one of the incomplete sample set of the imaging procedure. However, the incomplete sample sets and the sample source images may be combined in any way (for example, each incomplete sample set with one or more sample source images of the other incomplete sample sets, each sample source image with one or more other incomplete sample sets, using all or only parts of the incomplete sample sets and of the sample source images each of them one or more times, and so on) and each complete sample set may comprise any number of sample source images (simulated from one or more other incomplete sample sets).

In an embodiment, the method comprises training (by the computing system) the machine learning model to optimize a capability thereof to generate the sample target image of each of the complete sample sets from the sample baseline image and the sample source image of the complete sample set. However, the machine learning model may be trained in any way (for example, by selecting any training/verification sets from the complete sample sets, using any algorithm, such as Stochastic Gradient Descent, Real-Time Recurrent Learning, higher-order gradient descent, Extended Kalman-filtering and the like, any loss function, such as based on Mean Absolute Error, Mean Square Error, perceptual loss, adversarial loss and the like, defined at the level of the locations individually or of groups thereof, for a single upscaling factor, for multiple upscaling factors corresponding to the sample source images of each complete sample set, for a variable upscaling factor being a parameter of the operative machine learning model and so on); moreover, the training may be based on any additional information, down to none (for example, any number of complete sample sets formed by sample images being all acquired, either provided already formed or created from corresponding sample images, further sample images in each of one or more complete sample sets being acquired and/or simulated, such as corresponding to different doses of the contrast agent and/or different acquisition conditions, health conditions of the body-parts, type of the subjects and so on).

In an embodiment, the method comprises deploying (by the computing system) the machine learning model being trained. However, the machine learning model may be deployed in any way to any number and type of imaging systems (for example, distributed together with corresponding new imaging systems or for upgrading imaging systems already installed, put online and so on).

In an embodiment, the deployed machine learning model is for use in the medical imaging applications to mimic an increase of a dose of the contrast agent being administered to corresponding patients according to an upscaling factor corresponding to an inverse of the downscaling factor. However, the patients may be of any type (for example, persons, animals and so on); the machine learning model may be used to mimic the increase of the dose of the contrast agent in any way (for example, in real-time, off-line, locally, remotely and so on), with the upscaling factor corresponding to the inverse of the downscaling factor in any way (for example, equal to it, lower or higher than it, such as according to a corresponding multiplicative factor, and so on).

Further embodiments provide additional advantageous features, which may however be omitted at all in a basic implementation. In this respect, it is expressly intended that the features of each of the following embodiments may be combined with the above features either alone or in combination with the features of any number of the other following embodiments.

In an embodiment, the sample source-dose is comprised between the sample baseline-dose and the sample target-dose. However, the sample baseline-dose may have any value (either in absolute or in relative terms).

In an embodiment, the sample target-dose is a full-dose of the contrast agent being standard in clinical practice. However, the full-dose may be of any type (for example, for each type of medical imaging applications, fixed, depending on the type of the body-parts, on the type, weight, age and the like of the patients, and so on).

In an embodiment, at least part of the subjects are animals. However, the animals may be any percentage of the subjects (from none to all) and of any type (for example, rats, pigs and so on).

In an embodiment, the patients are persons. However, the persons may be of any type (such as gender, age, health condition and so on).

In an embodiment, the method comprises receiving (by the computing system) a plurality of sample images for each of the imaging procedures. However, the sample images may be in any number and received in any way (see above with reference to the incomplete sample sets).

In an embodiment, the sample images of each imaging procedure comprise one or more of the sample baseline images and one or more of the sample target images. However, the sample images may comprise any number of sample baseline images and sample target images.

In an embodiment, the method comprises creating (by the computing system) the incomplete sample sets of each imaging procedure based on the sample images of the imaging procedure. However, the incomplete sample sets may be created in any way (for example, based on the sample images as received or increased/decreased, combining each sample baseline image with one or more sample target images, each sample target image with one or more sample baseline images, using all or only parts of the sample baseline images and of the sample target images each of them one or more times, and so on).

In an embodiment, the method comprises generating (by the computing system) new one or more of the sample baseline images and/or of the sample target images for each of one or more of the imaging procedures from the corresponding sample baseline images and/or sample target images, respectively, being received. However, the new sample images may be generated in any number (for only the sample baseline images, only the sample target images or both of them) and in any way (for example, with each new sample image obtained by combining linearly/non-linearly two or more corresponding sample images, by adding any noise to a corresponding sample image, by using any autoencoder and so on) for any imaging procedures (from none to all).

In an embodiment, the method comprises generating (by the computing system) the new sample baseline images and/or new sample target images for each imaging procedure to equalize a number of the sample baseline images and a number of the sample target images of the imaging procedure. However, the new sample (baseline/target) images may be generated for any purpose (for example, to increase the sample baseline/target images in fewer number or both of them, to equalize their number, to obtain the required multiple sample baseline/target images from a single one of them, and so on).

In an embodiment, the method comprises discarding (by the computing system) one or more of the sample baseline images and/or of the sample target images for each of one or more of the imaging procedures. However, the sample images may be discarded in any number (for only the sample baseline images, only the sample target images or both of them) and in any way (for example, according to their quality level, diversity and so on) for any imaging procedures (from none to all).

In an embodiment, the method comprises discarding (by the computing system) the sample baseline images and/or sample target images for each imaging procedure to equalize a number of the sample baseline images and a number of the sample target images of the imaging procedure. However, the sample (baseline/target) images may be discarded for any purpose (for example, to decrease the sample baseline/target images in higher number or both of them, to equalize their number, to obtain the required multiple sample baseline/target images, to ensure a minimum quality level or diversity of the sample images, and so on).

In an embodiment, the method comprises creating (by the computing system) the incomplete sample sets of each of one or more of the imaging procedures by combining a single one of the sample baseline images with each of a plurality of the sample target images of the imaging procedure. However, this procedure may be applied to any number of imaging procedures (from none to all).

In an embodiment, the method comprises creating (by the computing system) the incomplete sample sets of each of one or more of the imaging procedures by combining a same number of the sample baseline images and of the sample target images of the imaging procedure biunivocally. However, this procedure may be applied to any number of imaging procedures (from none to all).

In an embodiment, the method comprises creating (by the computing system) the incomplete sample sets of each of one or more of the imaging procedures by combining the sample baseline images and the sample target images of the imaging procedure in all possible ways. However, this procedure may be applied to any number of imaging procedures (from none to all).

In an embodiment, the method comprises generating (by the computing system) the complete sample sets for each of one or more of the imaging procedures by combining a half of the incomplete sample sets and the sample source images being simulated from another half of the incomplete sample sets of the imaging procedure biunivocally. However, this procedure may be applied to any number of imaging procedures (from none to all).

In an embodiment, the method comprises generating (by the computing system) the complete sample sets for each of one or more of the imaging procedures by combining the incomplete sample sets and the sample source images being simulated from other ones of the incomplete sample sets of the imaging procedure in all possible ways. However, this procedure may be applied to any number of imaging procedures (from none to all).

In an embodiment, the method comprises generating (by the computing system) the complete sample sets for each of one or more of the imaging procedures by combining the incomplete sample sets and the sample source images being simulated from other ones of the incomplete sample sets of the imaging procedure biunivocally. However, this procedure may be applied to any number of imaging procedures (from none to all).

In an embodiment, each of the sample baseline images, each of the sample target images and each of the sample source images comprise a plurality of sample baseline values, of sample target values and of sample source values, respectively. However, the sample baseline/source/target values may be in any number and of any type (for example, real/complex numbers, with any range, in gray scale or colors, and so on).

In an embodiment, the method comprises calculating (by the computing system) each of the sample source values of each of the sample source images by applying a simulation formula depending on the downscaling factor. However, the simulation formula may be of any type (for example, linear, quadratic, cubic, function of the corresponding sample baseline value and/or sample administration value, and so on).

In an embodiment, the simulation formula is derived from a signal law expressing a magnitude of a response signal of the body-parts as a function of a local concentration of the contrast agent. However, the signal law may be of any type (for example, based on any extrinsic/intrinsic acquisition parameters and so on) and the simulation formula may be derived from the signal law in any way (for example, from any approximation of the signal law, the actual signal law and so on).

In an embodiment, the simulation formula is derived from the signal law being linearized with respect to the local concentration of the contrast agent. However, the signal law may be linearized in any way (for example, with any series expansion, any approximation, assuming any linear/non-linear relationship between the local concentration and the dose of the contrast agent, and so on).

In an embodiment, the simulation formula is derived from the signal law assuming a direct proportionality between the local concentration and a dose of the contrast agent. However, the direct proportionality between the local concentration and the dose of the contrast agent may be based on any proportionality factor.

In an embodiment, the sample baseline values, the sample target values and the sample source values are representative of the response signal of corresponding locations of the body-parts. However, the response signal may be represented in any way (for example, in magnitude form, in complex form, in positive/negative form and so on).

In an embodiment, the method comprises modulating (by the computing system) the downscaling factor to be used to calculate each of the sample source values of each of the sample source images according to an indication of the local concentration of the contrast agent in the corresponding location derived from the corresponding sample target value. However, the local concentration may be derived in any way (for example, set to the corresponding local contrast enhancement, calculated from the sample target value according to the signal law and so on) and the downscaling factor may be modulated according to any linear/non-linear function thereof (for example, according to a modulation factor determined empirically, calculated by using average/local values of any acquisition parameters and so on), down to be maintained always the same.

In an embodiment, the method comprises injecting (by the computing system) an artificial noise into each of the sample source images. However, the artificial noise may be of any type (for example, depending on the downscaling factor, fixed, and so on) and it may be injected into the sample source image in any way (for example, in additive form or in multiplicative form, such as at the level of each cell, group of cells and the like, in convolutional form, such as in circular or non-circular way with any stride, padding and the like, into the sample source image in magnitude form, in complex form, in k-space form, everywhere, only where the contrast agent is present and so on), down to none.

In an embodiment, the artificial noise has a statistical distribution depending on the downscaling factor. However, the statistical distribution of the artificial noise may be of any type (for example, normal with any mean value, Rayleigh, Rician and so on) and it may be obtained in any way (for example, by calculating artificial values of one or more statistical parameters, such as standard deviation, variance, skewness and the like, by applying any linear/non-linear function to reference values of the statistical parameters, such as obtained from the noise of the sample baseline image and the noise of the sample target image, only from the noise of the sample baseline image, only from the noise of the sample target image and the like, with the artificial values of the statistical parameters that may be then corrected heuristically, such as in the same way for all the statistical parameters or differently for each statistical parameter, by incrementing/decrementing them, according to any linear/non-linear function and so on).

In an embodiment, the method comprises training (by the computing system) a further machine learning model to optimize a capability thereof to generate the sample source image of each of at least part of the complete sample sets from the corresponding sample baseline image and sample target image. However, the further machine learning model may be of any type and it may be trained in any way (for example, either the same or different with respect to the machine learning model) by using any sample sets (for example, all of them after the completion of the incomplete sample sets analytically, only the sample sets provided already completed and so on).

In an embodiment, the method comprises generating (by the computing system) a refined version of each of the sample source images by applying the sample baseline image and the sample target image of the corresponding incomplete sample set to the further machine learning model being trained. However, the possibility is not excluded of using the training machine learning model in a different way (for example, to refine the sample source images of the incomplete sample sets, to generate them directly and so on).

In an embodiment, the method comprises repeating (by the computing system) said step of simulating the sample source images, said step of generating the complete sample sets and said step of training the machine learning model for a plurality of values of the downscaling factor. However, the values of the downscaling factor may be in any number and of any type (for example, distributed uniformly, with variable pitch, such as decrementing for incrementing values, and so on) and these steps may be repeated in any way (for example, consecutively, at different times and so on).

In an embodiment, the method comprises deploying (by the computing system) the machine learning model in corresponding configurations being trained with the values of the downscaling factor for selecting one or more corresponding values of the upscaling factor in each of the medical imaging applications. However, the different configurations may be deployed in any way (for example, all together, added over time and so on) and in any form (for example, corresponding configurations of a single operative machine learning model, corresponding instances of the operative machine learning model and so on), and they may be used for selecting the values of the upscaling factor in any number and in any way (for example, in discrete mode, in continuous mode, either the same or different with respect to the values of the downscaling factor, and so on).

In an embodiment, the machine learning model is a neural network. However, the neural network may be of any type (for example, an autoencoder, a multi-layer perceptron network, a recurrent network, a generative adversarial network and the like, shallow or deep with any number of layers, with any connections between layers, receptive field, stride, padding, activation functions and so on).

An embodiment provides a method of using the machine learning model being trained as above in a medical imaging application for imaging a body-part of a patient. However, the body-part may be of any type, in any condition and it may belong to any patient (see above). In any case, although the method may facilitate the task of a physician, it only provides intermediate results that may help him/her but with the medical activity stricto sensu that is always made by the physician himself/herself.

In an embodiment, the method comprises the following steps under the control of a computing system. However, the computing system may be of any type (see below).

In an embodiment, the method comprises receiving (by the computing system) one or more operative administration images being representative of the body-part of the patient to which the contrast agent has been administered at an operative administration-dose. However, the operative administration images may be in any number and of any type (for example, either the same or different with respect to the sample images) and they may be received in any way (for example, in real-time, off-line, locally, remotely and so on); moreover, the operative administration-dose may have any value (for example, either the same as or different from the sample source-dose, lower than, equal to or higher than the full-dose of the contrast agent, and so on). In any case, the contrast agent may have been administered to the patient in any manner, comprising in a non-invasive manner (for example, orally for imaging the gastrointestinal tract, via a nebulizer into the airways, via topical spray application) and in any case without any substantial physical intervention on the patient that would require professional medical expertise or entail any health risk for him/her (for example, intramuscularly).

In an embodiment, the method comprises receiving (by the computing system) at least one operative baseline image being representative of the body-part of the patient without the contrast agent or to which the contrast agent has been administered at an operative baseline-dose lower than the operative administration-dose. However, the operative baseline images may have been acquired in any way ensuring that no contrast agent significantly affects its content or after administration of the contrast agent at any operative baseline-dose (for example, either the same or different with respect to the source baseline image).

In an embodiment, the method comprises simulating (by the computing system) corresponding operative simulated images from the operative baseline image and the operative administration images with the machine learning model. However, the operative simulated images may be simulated in any way (for example, operating in any domain, such as magnitude, complex, k-space and the like, in real-time, off-line, locally, remotely and so on).

In an embodiment, the operative simulation images are representative of the body-part of the patient mimicking administration thereto of the contrast agent at an operative simulation-dose higher than the operative administration-dose (with a ratio between the operative simulation-dose and the operative administration-dose equal to an upscaling factor corresponding to an inverse of the downscaling factor of the machine learning model). However, the operative simulation-dose may have any value (for example, either the same as or different from the sample target-dose, lower than, equal to or higher than the full-dose, and so on).

In an embodiment, the method comprises outputting (by the computing system) a representation of the body-part based on the operative simulation images. However, the representation of the further body-part may be of any type (for example, the operative simulation images, the corresponding operative combined images and so on) and it may be output in any way (for example, displayed on any device, such as a monitor, virtual reality glasses and the like, or more generally output in real-time or off-line in any way, such as printed, transmitted remotely and so on).

Generally, similar considerations apply if the same solution is implemented with an equivalent method, provided that it remains within the scope of the claims (by using similar steps with the same functions of more steps or portions thereof, removing some non-essential steps or adding further optional steps); moreover, the steps may be performed in a different order, concurrently or in an interleaved way (at least in part).

An embodiment provides a computer program, which is configured for causing a computing system to perform the method of above when the computer program is executed on the computing system. An embodiment provides a computer program product, the computer program product comprising one or more non-transitory computer readable storage media having program instructions collectively stored on the readable storage media, the program instructions readable by a computing system to cause the computing system to perform the same method. However, the (computer) program may be executed on any computing system (see below). The program may be implemented as a stand-alone module, as a plug-in for a pre-existing software program (for example, a configuration application or an imaging application) or even directly in the latter.

Generally, similar considerations apply if the program is structured in a different way, or is additional modules or functions are provided (provided that it remains within the scope of the claims). Particularly, the program may take any form suitable to be used by the computing system, thereby configuring it to perform the desired operations; the program may be in the form of external or resident software, firmware, or microcode (either in object code or in source code), for example, to be compiled or interpreted. Moreover, it is possible to provide the program on any computer readable storage medium. The storage medium is any tangible medium (different from transitory signals per se) that may retain and store instructions for use by the computing system. For example, the storage medium may be of electronic, magnetic, optical, electromagnetic, infrared, or semiconductor type; examples of such storage medium are fixed disks (where the program may be pre-loaded), removable disks, memory keys (for example, USB), and the like. The program may be downloaded to the computing system from the storage medium or via a network (for example, the Internet, a wide area network and/or a local area network comprising transmission cables, optical fibers, wireless connections, network devices); one or more network adapters in the computing system receive the program from the network and forward it for storage into one or more storage devices of the computing system. In any case, the solution according to an embodiment of the present disclosure lends itself to be implemented even with a hardware structure (for example, by electronic circuits integrated on one or more chips of semiconductor material), or with a combination of software and hardware suitably programmed or otherwise configured. An embodiment provides a computing system, which comprises means configured for performing the steps of the method of above. An embodiment provides a computing system comprising a circuit (i.e., any hardware suitably configured, for example, by software) for performing each step of the same method. However, the computing system may be of any type (for example, the configuration computer implemented by a server, a virtual machine, a cloud service and the like for training the machine learning model, the control computer of each scanner implemented by a PC, a control unit of each scanner and the like for using the machine learning model being trained).

Generally, similar considerations apply if the computing system has a different structure, comprises equivalent components or has other operative characteristics, provided that it remains within the scope of the claims. In any case, every component thereof may be separated into more elements, or two or more components may be combined together into a single element; moreover, each component may be replicated to support the execution of the corresponding operations in parallel. Moreover, unless specified otherwise, any interaction between different components generally does not need to be continuous, and it may be either direct or indirect through one or more intermediaries.

An embodiment provides a medical method applied to a body-part of a patient. However, the medical method may be applied to any body-part of any patient (see above).

In an embodiment, the medical method comprises acquiring an operative baseline image being representative of the body-part. However, the operative baseline image may be acquired in any way (for example, before administering the contrast agent, with administration of the contrast agent at an operative baseline-dose lower than the operative administration-dose and so on).

In an embodiment, the medical method comprises administering a contrast agent at an operative administration-dose to the patient. However, the contrast agent may be administered in any way (for example, with a syringe, an infusion pump, in advance, shortly before acquiring the operative administration images, continuously during their acquisition, and so on).

In an embodiment, the medical method comprises acquiring one or more operative administration images being representative of the body-part in response to said administering the contrast agent to the patient (corresponding operative simulation images being simulated from the operative baseline image and the operative administration images and a representation of the body-part based on the operative simulation images being output according to the method of above). However, the operative administration images may in any number and acquired in any way (for example, after the administration of the contrast agent with any delay, during the administration of the contrast agent, continually, at specific times and so on).

In an embodiment, the medical method comprises performing a medical procedure relating to the body-part according to the representation of the body-part. However, the medical procedure may be of any type (for example, a diagnostic procedure, a therapeutic procedure, a surgical procedure and so on).

1. A method for training a machine learning model for use in medical imaging applications, wherein the method comprises, under the control of a computing system:
providing to the computing system, a plurality of incomplete sample sets for each of a plurality of imaging procedures relating to corresponding body-parts of subjects, each of the incomplete sample sets comprising at least one sample target image being representative of the corresponding body-part of the subject to which a contrast agent has been administered at a sample target-dose and at least one sample baseline image being representative of the corresponding body-part of the subject without the contrast agent or to which the contrast agent has been administered at a sample baseline-dose lower than the sample target-dose,
simulating, by the computing system a plurality of sample source images, at least one of the sample source images being simulated from each of at least part of the incomplete sample sets of each of the imaging procedures to be representative of the corresponding body-part mimicking administration to the corresponding subject of the contrast agent at a sample source-dose lower than the sample target-dose with a ratio between the sample source-dose and the sample target-dose equal to a downscaling factor,
generating, by the computing system, one or more complete sample sets for each of the imaging procedures, each of the complete sample sets comprising the sample baseline image and the sample target image of one of the incomplete sample sets of the imaging procedure and the sample source image being simulated from another one of the incomplete sample set of the imaging procedure,
training, by the computing system, the machine learning model to optimize a capability thereof to generate the sample target image of each of the complete sample sets from the sample baseline image and the sample source image of the complete sample set, and
deploying, by the computing system, the machine learning model being trained for use in the medical imaging applications to mimic an increase of a dose of the contrast agent being administered to corresponding patients according to an upscaling factor corresponding to an inverse of the downscaling factor.

2. The method according to claim 1, wherein the sample source-dose is comprised between the sample baseline-dose and the sample target-dose.

3. The method according to claim 1, wherein the target-dose is a full-dose of the contrast agent being standard in clinical practice.

4. The method according to claim 1, wherein at least part of the subjects are animals and wherein the patients are persons.

5. The method according to claim 1, wherein the method comprises:
receiving, by the computing system, a plurality of sample images for each of the imaging procedures comprising one or more of the sample baseline images and one or more of the sample target images, and
creating, by the computing system, the incomplete sample sets of each imaging procedure based on the sample images of the imaging procedure.

6. The method according to claim 5, wherein the method comprises:
generating, by the computing system new one or more of the sample baseline images and/or of the sample target images for each of one or more of the imaging procedures from the corresponding sample baseline images and/or sample target images, respectively, being received to equalize a number of the sample baseline images and a number of the sample target images of the imaging procedure.

7. (canceled)

8. The method according to claim 5, wherein the method comprises:
discarding, by the computing system, one or more of the sample baseline images and/or of the sample target images for each of one or more of the imaging procedures to equalize a number of the sample baseline images and a number of the sample target images of the imaging procedure.

9. (canceled)

10. The method according to claim 1, wherein the method comprises:
creating, by the computing system, the incomplete sample sets of each of one or more of the imaging procedures by combining:
a single one of the sample baseline images with each of a plurality of the sample target images of the imaging procedure,
a same number of the sample baseline images and of the sample target images of the imaging procedure biunivocally, or
the sample baseline images and the sample target images of the imaging procedure in all possible ways.

11. The method according to claim 1, wherein the method comprises:
generating, by the computing system, the complete sample sets for each of one or more of the imaging procedures by combining:
a half of the incomplete sample sets and the sample source images being simulated from another half of the incomplete sample sets of the imaging procedure biunivocally,
the incomplete sample sets and the sample source images being simulated from other ones of the incomplete sample sets of the imaging procedure in all possible ways, or
the incomplete sample sets and the sample source images being simulated from other ones of the incomplete sample sets of the imaging procedure biunivocally.

12. The method according to claim 1, wherein each of the sample baseline images, each of the sample target images and each of the sample source images comprise a plurality of sample baseline values, of sample target values and of sample source values, respectively, the method comprising:
calculating, by the computing system, each of the sample source values of each of the sample source images by applying a simulation formula depending on the downscaling factor, the simulation formula being derived from a signal law expressing a magnitude of a response signal of the body-parts as a function of a local concentration of the contrast agent being linearized with respect to the local concentration of the contrast agent.

13. (canceled)

14. The method according to claim 12, wherein the simulation formula is derived from the signal law assuming a direct proportionality between the local concentration and a dose of the contrast agent.

15. The method according to claim 12, wherein the sample baseline values, the sample target values and the sample source values are representative of the response signal of corresponding locations of the body-parts, the method comprising:
modulating, by the computing system, the downscaling factor to be used to calculate each of the sample source values of each of the sample source images according to an indication of the local concentration of the contrast agent in the corresponding location derived from the corresponding sample target value.

16. The method according to claim 1, wherein the method comprises:
injecting, by the computing system, an artificial noise into each of the sample source images, the artificial noise having a statistical distribution depending on the downscaling factor.

17. The method according to claim 1, wherein the method comprises:
training, by the computing system, a further machine learning model to optimize a capability thereof to generate the sample source image of each of at least part of the complete sample sets from the corresponding sample baseline image and sample target image, and
generating, by the computing system, a refined version of each of the sample source images by applying the sample baseline image and the sample target image of the corresponding incomplete sample set to the further machine learning model being trained.

18. The method according to claim 1, wherein the method comprises:
repeating, by the computing system, said simulating the sample source images, said generating the complete sample sets and said training the machine learning model for a plurality of values of the downscaling factor, and
deploying, by the computing system, the machine learning model in corresponding configurations being trained with the values of the downscaling factor for selecting one or more corresponding values of the upscaling factor in each of the medical imaging applications.

19. The method according to claim 1, wherein the machine learning model is a neural network.

20. A method of using the machine learning model being trained with the method according to claim 1 in a medical imaging application for imaging a body-part of a patient, wherein the method comprises, under the control of a computing system:
receiving, by the computing system, one or more operative administration images being representative of the body-part of the patient to which a contrast agent has been administered at an operative administration-dose and at least one operative baseline image being representative of the body-part of the patient without the contrast agent or to which the contrast agent has been administered at an operative baseline-dose lower than the operative administration-dose,
simulating, by the computing system, corresponding operative simulated images from the operative baseline image and the operative administration images with the machine learning model, the operative simulation images being representative of the body-part of the patient mimicking administration thereto of the contrast agent at an operative simulation-dose higher than the operative administration-dose with a ratio between the operative simulation-dose and the operative administration-dose equal to an upscaling factor corresponding to an inverse of the downscaling factor of the machine learning model, and
outputting, by the computing system, a representation of the body-part based on the operative simulation images.

21. (canceled)

22. A computer program product, the computer program product comprising one or more non-transitory computer readable storage media having program instructions collectively stored on the readable storage media, the program instructions readable by a computing system to cause the computing system to perform the method according to claim 1.

23. (canceled)

24. A computing system comprising a circuit for performing each step of the method according to claim 1.

25. A medical method applied to a body-part of a patient, wherein the medical method comprises:
acquiring at least one operative baseline image being representative of the body-part,
administering a contrast agent at an operative administration-dose to the patient,
acquiring one or more operative administration images being representative of the body-part in response to said administering the contrast agent to the patient, corresponding operative simulation images being simulated from the operative baseline image and the operative administration images and a representation of the body-part based on the operative simulation images being output according to the method of claim 20, and
performing a medical procedure relating to the body-part according to the representation of the body-part.
